(19) United States
(12) Patent Application Publication
(10) Pub. No.: US 2011/0045001 A1
(43) Pub. Date: Feb. 24, 2011

(54) Transfection results of non-viral gene delivery systems by influencing of the innate immune system

(75) Inventors: Roland Klosel, Munchen (DE); Stephan Konig, Munchen (DE)

Correspondence Address: EDWARDS ANGELL PALMER & DODGE LLP, P.O. BOX 55874, BOSTON, MA, 02205, US

(73) Assignee: Biontex Laboratories GmbH, Martinsried/Planegg (DE)

(21) Appl. No.: 12/412,550

(22) Filed: Mar. 27, 2009

Related U.S. Application Data
(60) Provisional application No. 61/072,135, filed on Mar. 28, 2008.

(51) Int. Cl. A61K 39/395; A61P 25/00; A61P 35/00; A61P 31/12; A61P 29/00; A61K 48/00; C12N 15/87

(52) U.S. Cl. 424/172.1; 514/44 R; 435/455; 435/461

(57) ABSTRACT

The innate immune system of eukaryotes is able to recognise foreign genetic material by means of Toll-like receptors and to initiate signal transduction cascades that trigger an antiviral state of cell populations by way of an interferon response. That antiviral state is also a barrier for non-viral gene delivery systems. If the signal transduction cascade is interrupted intracellularly or intercellularly, transfection efficiencies of non-viral gene delivery systems can be increased and undesirable changes in the expression profile can be avoided. Since RNA-interference is to be attributed to the antiviral state, the RNAi machinery is likewise activated after activation of the innate immune system. In that way, knock-down efficiencies on transfection with siRNA can be increased.

### PRIOR ART

When the body exhibits immune responses (Luke A. et al.; Spektrum der Wissenschaft, August 2005, pages 68-75) to an infectious or immunological challenge, a distinction is drawn between the innate immune response (innate immunity) and the acquired immune response (antigen-specific acquired immunity).

Acquired immunity is developed only in the event of infection with pathogens. It has a kind of memory so that a second infection caused by the same causative organism generally does not result in an outbreak of the illness. It is on that principle that vaccines are based. If only acquired immunity existed, the organism would be totally unprotected against the first infection. That is not the case, however, because a further very original immunity exists which is referred to as innate immunity and is found in organisms ranging from the fly Drosophila to mammals, and indeed is found even in plants.

Innate immunity is the first line of defence against pathogens and is a very old system in evolutionary terms. In the case of innate immunity, the disease-associated molecular patterns, so-called pathogen-associated molecular patterns (PAMPs), are recognised by means of so-called Toll-like receptors (TLRs) (Heine H. et al., Int. Arch. Allergy Immunol. 2003; 130; 180-192 and Uematsu S. et al., J. Biol. Chem. 2007, May 25; 282 (21); 15319-23) and RIG-I-like helicases (RLHs), and appropriate inflammatory and immune reactions are initiated. As a result, the organism is able to distinguish between “itself” and “not itself”. RLHs are expressed ubiquitously in cytosol, where they are capable of recognising the dsRNA that is formed in the case of viral infection. TLRs and RLHs belong to a group of receptors also referred to as pattern recognition receptors (PRRs).

Toll-Like Receptors (TLRs)

Toll-like receptors were first discovered in the mid-1990s (Zimmer A. et al.; PNAS, 1999; 96(10), 5780-5785). The name is derived from a protein found in Drosophila Melanogaster by Christiane Nüsslein-Volhard, which she named “Toll”. TLR proteins resemble that type and are therefore referred to as “Toll-like” proteins. They are transmembrane proteins having an extracellular, “leucine-rich repeat” domain (LRR) and also a cytoplasmic domain which is homologous to that of the IL-1R family. The different TLRs react selectively to different molecular viral and bacterial components and, via a signal transduction cascade, control corresponding activation of genes. That happens in the first instance by way of so-called adapter molecules and subsequently by way of kinases which finally activate transcription factors (for example NF-kB and the IRF families) by phosphorylation thereof or corresponding intracellular inhibitors of those transcription factors. Finally, in addition to a large number of specific genes having antimicrobial action, so-called cytokines are produced. Cytokines are in turn necessary stimulators for acquired immunity and are accordingly also a link between innate and acquired immunity. The principles of ligand recognition, signal transduction and signal transmission are, however, known only rudimentarily.

Thirteen different TLRs are known hitherto (ten of them in human beings), their number being sufficient for recognition of all pathogenic causative organisms, ranging from bacteria through fungi to the viruses. The receptors recognise structures common to all causative organisms, and furthermore occasionally also a number of constituents simultaneously, without the latter being structurally similar. For example, TLR4 recognises lipopolysaccharides but also taxol. It has not been known hitherto how TLRs are able to do this. TLRs differ only slightly from species to species.

The following groups of molecules have been known hitherto as ligands of TLRs which result in a triggering of signal transduction cascades:

TLR1:

forms a heterodimer with TLR2, is the receptor of triacylated lipoprotein and zymosan from yeasts.

TLR2: is the receptor for certain peptidoglycans, lipopeptides, glycolipids and various bacteria.

TLR3:

recognises long dsRNA, as occurs in the case of virus replication in infected cells.

TLR4:

is the receptor for lipopolysaccharides (LPS, also endotoxins), various coat glycoproteins (also of viruses) and taxol. LPS are constituents of bacterial cell walls. The TLR4 receptor requires for its function an additional membrane-bound protein (TLR assisting protein): CD14, for example, binds the LPS and supplies it to the TLR4 receptor, the binding to CD14 alone not triggering a signal transduction cascade.

TLR5:

is the receptor of flagellin, a main constituent of the cilia (flagellae) with which bacteria move.

TLR6:

forms a heterodimer with TLR2, is the receptor of diacylated lipoprotein and certain peptidoglycans. A special lipoprotein (MALP-2=macrophage-activating lipopeptide) is detected by means of the assistance of the membrane-bound protein CD36 (TLR assisting protein).

TLR7 & TLR8:

are receptors for imidazoquinolines and of ssRNA/dsRNA, for example of RNA viruses.

TLR9:

is the receptor for bacterial DNA, or for non-methylated CpG motifs, which occurs in large numbers in bacterial DNA (20× more frequently than in mammalian cells). The CpG motif in mammalian cells is highly methylated, with the result that it can be distinguished. What applies to bacterial DNA is similarly true of viral DNA which is also detected by TLR9. The immunostimulatory property of bacterial DNA was reported as early as the beginning of the 1980s by the group led by Dr. Tokunaga. The group led by Dr. Shizuo Akira identified the TLR9 receptor as associated receptor (clarification of the roles of Toll-receptors and their signal transduction cascades by means of gene-targeting, Robert Koch lecture by Dr. Shizuo Akira, General Press Information 2002; www.robert-koch.stiftung.de).

TLR10:

Ligand not yet known.

TLR11:

is receptor for the uropathogenic bacterium Escherichia coli and the profilin-like protein of the protozoan Toxoplasma gondii.

TLR12:

Function and ligand still unknown.

TLR13:

Function and ligand still unknown.

Localisation of the TLR:

TLR2, 4, 5 and 6 are located especially in the plasma membranes of monocytes, natural killer cells, mast cells or myeloid dendritic cells, while 7, 8 and 9 are located especially in endosomes of immune cells (Siegmund-Schultze N., www.aerzteblatt.de). The activation of the immune response therefore requires intracellular uptake by way of endocytosis and maturation of the endosomes. The signal transmission begins here in an endosomal compartment. In the case of TLR 3 there are indications that it is located in the plasma membranes, but there are also descriptions in the literature which assume an endosomal localisation. TLRs frequently act in pairs and occur in various cell types in various combinations.

Signal Transduction Cascades:

Although the signal transduction pathways of the various TLRs (Perry A. K. et al., Cell Research 2005; 15(6); 407-422 and Kawai T. et al., J. Biochem.; 2007; 141; 137-145) have some similarities, they also definitely exhibit relatively large differences, ultimately resulting in different gene expression and thus different biological reactions. With the exception of TLR3, all TLRs transmit their signal to the adapter protein MyD88. MyD88 plays a crucial role in signal transmission by way of the TLR/interleukin-1 receptor. The cytosolic domain of the TLRs exhibits great similarity to that of the interleukin-1 receptor and is therefore also referred to as the Toll/IR-1 receptor domain (TIR). MyD88-deficient splenocytes, for example, exhibited no reactions to interleukin-1, LPS or CpG-DNA. In addition, in the case of MyD88-deficient cells, no activation of signal molecules, such as NF-kB or MAP kinases, was observed in reaction to TLR2, TLR7, TLR9 ligands. That is a significant pointer to the compete dependence of the TLRs (except for TLR3) on MyD88 for their signal transmission. Other adapter molecules are, for example, TIRAP (Toll-Interleukin-1 Receptor(TIR)-domain-containing adapter protein (TLR1, TLR2, TLR4 and TLR6), Mal (MyD88-adapter-like), TRIF (TLR3 and TLR4) and TRAM (TLR4).

Which proteins, in addition to the adapter molecules, also play a part depends upon the TLR in question. Presented in general, simplified terms, a signal transduction cascade usually begins with a receptor at the cell surface having a cytosolic domain, which receptor, on being loaded with a suitable ligand, transmits its signal by way of cytosolic adapter molecules to kinases which activate transcription factors via cascades. The activated transcription factors are localised in the nucleus and trigger the expression of proteins, mostly cytokines.

Signal Transduction Cascade via TLR1/TLR2, TLR2/TLR2, TLR2/TLR6

The receptors that occur in the pairs in question, on being loaded with suitable ligands, trigger the same signal transduction cascades. Ultimately there are activated inter alia the transcription factors NF-kB and AP-1 which especially result in expression of cytokines, the adapter molecules RAC-1, TIRAP, MyD88 and TRAF6 being involved in the signal transmission. Kinases involved are at least IRAK1, IRAK4, TAK1, PI 3K, IKKalpha, IKKbeta, IKKgamma, JNK, p38 MAPK and MKKs.

Signal Transduction Cascade via TLR4

The receptor requires the membrane-bound protein CD14 to function fully. CD14 binds corresponding agonists and supplies them to the receptor. That receptor, on being loaded with suitable ligands, ultimately triggers the activation of the transcription factors NF-kB, AP-1, IRF3 and IRF7 and results, in turn, especially in expression of cytokines, the adapter molecules TIRAP, MyD88, TRAM, TRIF, TRAF3, TRAF6, NAP1 and RIP1 being involved in the signal transmission. Kinases involved are at least IRAK1, IRAK4, TAK1, IKKalpha, IKKbeta, IKKgamma, IKKepsilon, TBK1, ERK1, ERK2, JNK, p38 MAPK, MEK1, MEK2 and MKKs.

Signal Transduction Cascade via TLR5

The receptor, on being loaded with suitable ligands, ultimately triggers the activation of the transcription factors NF-kB and AP-1, which result especially in expression of cytokines, the adapter molecules MyD88 and TRAF6 being involved in the signal transmission. Kinases involved are at least IRAK1, IRAK4, TAK1, IKKalpha, IKKbeta, IKKgamma, JNK, p38 MAPK and MKKs.

Signal Transduction Cascade via TLR10, 11, 12, 13

The receptors, on being loaded with suitable ligands, ultimately trigger the activation of the transcription factors NF-kB and AP-1, which result especially in expression of cytokines, the adapter molecules MyD88 and TRAF6 being involved in the signal transmission. Kinases involved are at least IRAK1, IRAK4, TAK1, IKKalpha, IKKbeta, IKKgamma and MKKs.

Signal Transduction Cascade via TLR3:

The receptor, on being loaded with suitable ligands, ultimately triggers the activation of the transcription factors NF-kB, AP-1, IRF3 and IRF7, there again being increased expression especially of cytokines, the adapter molecules TRIF, TRAF6, TRAF3, NAP1 and RIP1 being involved in the signal transmission. Kinases involved are at least IRAK1, IRAK4, TAK1, IKKalpha, IKKbeta, IKKgamma, IKKepsilon, TBK1, PKR, PI K3, JNK, p38 MAPK and MKKs.

Signal Transduction Cascades via TLR7, TLR8 and TLR9

The receptors, on being loaded with suitable ligands, ultimately trigger the activation of the transcription factors NF-kB, AP-1, IRF1, IRF5 and IRF7, there again being increased expression especially of cytokines, the adapter molecules MyD88, TRAF6 and TRAF3 being involved in the signal transmission. Kinases involved are IRAK1, IRAK4, TAK1, IKKalpha, IKKbeta, IKKgamma, JNK, p38 MAPK and MKKs.

TLRs are of great therapeutic interest. TLR agonists are used, for example, as adjuvants in vaccination strategies or in cancer treatment. Examples are the treatment of basal cell carcinoma by the TLR7/8 agonists imiquimod/resiquimod and the treatment of cancer of the bladder by a TLR2 agonist. The TLR9 receptor is activated by a synthetic CpG-containing oligonucleotide (CpG 7909 and CpG 10101) for the treatment of auto-immune diseases, cancer and infectious diseases. TLR9-based treatment strategies are commercially available from Coley Pharmaceuticals.

TLR7 and TLR8 Agonists

Known commercially available agonists for the TLR7 and TLR8 receptors are generally imidazoquinolines (Schon et al., Oncogene, 2008, 27, 190-199), such as imiquimod (R837, 1-(2-methylpropyl)-1H-imidazo[4,5-c]quinoline-4-amine), resiquimod (R848, 4-amino-2-(ethoxymethyl)-a,a-dimethyl-1H-imidazo[4,5-c]quinoline-1-ethanol) and gardiquimod (1-(4-amino-2-ethylaminomethylimidazo[4,5-c]quinolin-1-yl)-2-methylpropan-2-ol), guanosine analogues, such as, for example, loxoribine (7-allyl-7,8-dihydro-8-oxo-guanosine) and others such as, for example, bropirimine (2-amino-5-bromo-6-phenyl-4-pyrimidinone). Further imidazoquinolines have been described in the literature (Miller et al., Drug News & Perspectives, 2008, 21(2), 69-87; Gorden et al., J. Immunol., 2005, 174, 1259-1268; Jurk et al., Eur. J. Immunol., 36, 1815-1826; 2006; Gorden et al., J. Immunol., 2006, 177, 8164-8170) and are to be incorporated by reference. Further agonists, some of which are likewise commercially available, are thiazoloquinolines such as CL075 (Gordon K B et al., J. Immunol., 2005, 174(3), 1259-68) and CL097 (Schindler U. et al., Mol. Cell Biol., 1994, 14(9), 5820-5831) and the guanosine analogue isatoribine (7-thia-8-oxoguanosine) (Horsmans Y. et al., Hepatology, 42(3), 724-731).

Known agonists for TLR8 are quite generally ssRNA, especially single-stranded polyuridine and ssRNA having U-rich or GU-rich sequences (Diebold et al., Science, 2004, 303, 1529-1531; Heil et al., Science, 2004, 303, 1526-1529; Lund et al., Proc. Natl. Acad. Sci. USA, 2004, 101, 5598-5603), it being possible for the ssRNA also to be in phosphothioate form. Particular mention should be made of the sequence motifs UGUGU and GUCCUUCAA (Hornung et al., Nat. Med., 2005, 11, 263-270; Judge et al., Nat. Biotechnol., 2005, 23, 457-462) which are especially stimulating in ssRNA from a length of 16 bp. The ssRNAs must naturally be transported into the endosomes in order to reach the TLR8. As a rule, this is effected with the customary transfection reagents. InvivoGen sells an ssRNA40 complexed with a cationic lipid (LyoVec) as TLR8 agonist.

However, a large number of diseases can also be triggered by an overreaction of the innate immunity, for example autoimmune diseases such as rheumatic arthritis and systemic Lupus erythematodes. In this case TLRs react with degradation products of endogenous cells and thus misdirect the immunity.

TLRs are even suspected of having a causal connection with cardio-vascular disorders. Inflammatory reactions of the heart can contribute to the formation of arteriosclerotic plaques which can ultimately lead to infarction as a result of vessel blockage.

Cytokines:

Cytokines are multifunctional signal substances. They are sugar-containing proteins which have a regulating function for the growth and the differentiation of the cells of the body. Some of them are therefore also referred to as growth factors. Many cytokines also play an important role in immunological reactions and are therefore also referred to as mediators. Cytokines are released from the cells into the surrounding medium by secretion and stimulate other cells when those cells have a suitable receptor. Cytokines are divided into five main groups:

1. Interferons (IFN)

Interferons instruct cells to form proteins which prevent a viral infection or render such infection more difficult. Interferons can also have antitumoral activity.

2. Interleukins (IL)

Interleukins serve especially for communication between immune system cells and thus increase co-ordination in protection against pathogens and treatment of tumours.

3. Colony-Stimulating Factors

Colony-stimulating factors are formed in the kidney. They are growth factors for blood corpuscles.

4. Tumour Necrosis Factors (TNF)

The most important function of TNFs is to regulate the activity of various immune cells. They are secreted mainly by macrophages. TNFs are able to stimulate cell death (apoptosis), cell proliferation, cell differentiation and secretion of other cytokines.

5. Chemokines

Chemokines are chemo-attractants which cause cells having suitable receptors to migrate to the source of the chemokines by chemotaxis.

Interferons (IFN), especially interferons of type I, are of special importance as mediators of immunological processes. The first interferon of that kind was discovered by Isaacs and Lindemann in 1957 (Isaacs, A. et al.; J. Proc. R. Soc. Lond. B. Biol. Sci. 147, 258-267). The name originates from the fact that the protein interferes with the replication of viruses. Type I interferons are key cytokines which trigger an antiviral response by cells, establish an “antiviral state” and stimulate cells of the immune system to an antiviral response. That group binds to a receptor, the so-called IFN-alpha receptor (IFNAR), which consists of two protein chains (IFNAR1 and IFNAR2). Various sub-types are differentiated. They are referred to as IFN-alpha, IFN-beta, IFN-kappa, IFN-delta, IFN-epsilon, IFN-tau, IFN-omega and IFN-zeta. In turn, special importance is attached here to the interferons of types alpha and beta which are secreted by a large number of cells, for example inter alia by lymphocytes, macrophages, fibroblasts, endothelial cells and osteoplasts.

The IFN-alpha proteins occur in thirteen sub-types which are referred to as IFNAX (x=1, 2, 4, 5, 6, 7, 8, 10, 13, 14, 16, 17 and 21). All their genes are located cluster-like on chromosome 9.

Of the IFN-beta proteins, two have been described. They are IFNB1 and IFNB3. A protein described as IFNB2 was later identified as being a known interleukin.

By means of a signal transduction cascade—the so-called JAK/STAT pathway—after the binding to the interferon-type-1-receptor located in the outer cell membrane, the transcription factor “interferon stimulated gene factor 3” (ISGF3), a heterotrimer of the transcription factors STAT1, STAT2 and “IFN regulatory factor 9” (IRF9), is induced, which migrates into the cell nucleus and there induces the transcription of hundreds of effector molecules (via so-called IFN inducible genes). Those effector molecules directly influence protein synthesis, cell growth and survival in the process of establishing the so-called “antiviral state”. In that state the infectiousness of the viruses is protected against or at least reduced, for example by means of reduced replication rates.

In addition, the adaptive immune system is activated by triggering maturation of dendritic cells and activating the antibody response of the B cells and the T cell response. Lymphocytes and monocytes are recruited to the site of the infection by induced chemokines.

Stress too can initiate signal transduction pathways which lead to an antiviral state. Those signal transduction cascades intersect the signal transduction cascades of the TLR.

Stress signal transduction pathways:

cellular stress can be triggered by:
heat/cold
UV
mechanical stress/shearing forces
lack of oxygen
lack of nutrients
osmotic stress
oxidative stress/free radicals
inflammation
biological and chemical agents (for example TNFalpha, chemotherapeutics)

The cells react to stress with complex changes in the activity of signal chains in which specific MEK, MSK and MSAP kinases and various transcription factors (for example NH-kB), apoptosis regulators and cell cycle regulators are involved. GTP-binding proteins (Ras/Rho family) that are bound to the membrane play a special role in the reaction of the cells to cellular stress.

The innate immune response takes place both intracellularly and intercellularly. In the case of the intracellular response, a cell affected by contact with a pathogen triggers signal transduction cascades via PRRs, such as, for example, TLRs and RLHs, resulting in a change in the physiological state and the expression profile of the cell. Alongside there is an intercellular response in which the cell affected by contact with a pathogen “informs” other cells, which have not been exposed to direct contact with the pathogen in question, of the “infection” with the pathogen, the cell affected by contact with a pathogen releasing cytokines which are detected by cytokine receptors located on the other cells that have not come into contact with the pathogen. The binding of the cytokines to the cytokine receptors triggers a signal transduction cascade in the cells that have not been exposed to direct contact with the pathogen, with the result that their physiological state and expression profile is changed too, although they have not come into direct contact with the pathogen. The change in the physiological state and the expression profile of the cells is intended to protect against the pathogenic attack and ensure the survival of the cells.

The intercellular response is distinguished from the intracellular response by the different receptors and agonists. In the case of the intercellular response, cytokine receptors and, in the case of the intracellular response, PRRs act as receptors. The agonists of the intercellular response are cytokines and the agonists in the case of the intracellular response are pathogenic patterns.

Gene Delivery Methods

Transfection, that is to say the introduction of genetic material into eukaryotic cells, especially mammalian cells, is a method without which modern research is unimaginable nowadays (Domb A. J.; Review in Molecules; 2005; 10; 34 and Xiang G; Keun-Sik K.; Dexi L.; Review in The AAPS Journal; 2007; 9(1) Article 9; http://www.aapsj.org). Without such a method it would be substantially more difficult to clarify the function of various genes. Not to be forgotten is the possibility of using this method to prepare true-to-original proteins of eukaryotic origin, because the correct post-translational modification is ensured by the eukaryotic cells, unlike prokaryotic cells which were often used in the past. Furthermore, it is expected that in the near future particularly the introduction of genetic material into human cells, that is to say gene therapy, will become a part of modern medicine in the form of clinically tested procedures and treatments. The introduction of genetic material makes it possible, for example, in eukaryotic cells to replace destroyed DNA regions and thus to correct defective functions. Moreover, it is possible to insert suicide genes which, for example, force cancer cells to “commit suicide”. The knock-down of genes can also be achieved, however, by the use, for example, of siRNA (small interfering RNA), ribozymes or antisense molecules. The possibility of being able to access the genetic control apparatus of the cell therefore provides mankind with a valuable means of increasing its understanding of and also its influence on the naturally occurring processes in a cell.

Over past years, research into so-called gene delivery methods (gene delivery systems) that can be used both in vitro and in vivo has gained enormous importance, because they offer excellent prospects for achieving a breakthrough in gene therapy. One focus of interest of gene therapy research is the use of viruses as carrier systems. Because the introduction of DNA or RNA into foreign cells is an integral component of the replication cycle of viruses, that ability has been refined by a natural, evolutionary process in the history of the development of viruses to the extent that today there is no more effective gene carrier. The naturally occurring viruses are manipulated by genetic engineering in such a way that they lose their ability to reproduce and their pathogenicity, but are able to infect a cell with recombinantly introduced genetic material. Because viruses, apart from consisting of genetic material, consist substantially of proteins, however, they offer the immune system a large target for attack and at the same time the immune system, in a likewise evolutionary adaptation process, has developed strategies to defend itself against such invaders. The immune response of the body is therefore cited as a particularly significant factor in respect of failed gene therapy studies.

The gene delivery methods currently available can be divided into two main groups: viral systems and non-viral systems. The non-viral systems can in turn be divided into chemical methods and physical methods.

Of the non-viral systems that are based on chemical methods, special mention should be made of those which are based on cationic lipids (so-called lipofection) or cationic polymers (so-called polyfection). Their efficiency generally lies far behind that of viral systems.

Examples of well-known cationic polymers are poly-L-lysine (PLL), (EP 388758) polyethyleneimine (PEI), (J. P. Behr et al.; Proc. Natl. Acad. Sci. USA; 1995; 92; 7297; WO 9602655), diethylaminoethyl dextran (DEAE), (S. C. De Smedt et al., Phar. Res.; 2000; 17; 113), Starburst dendrimers (PAMAM), (F. C. Szoka et al., Bioconjug. Chem.; 1996; 7; 703; WO 9502397), chitosan derivatives (W. Guang Liu et al.; J. Control. Release; 2002; 83; 1) and also polydimethylaminoethyl methacrylates (P. van de Wetering et al.; J. Gene Med.; 1999; 1; 156; WO 9715680). The widely used Ca phosphate precipitation method also uses a “cationic polymer” in the broader sense and can therefore be included in this group.

Commercially available products of such cationic polymers are, for example, Superfect, Polyfect (Qiagen), ExGen500 (Biomol) and jetPEI (Qbiogene).

Similarly known cationic lipids (J. P. Behr; Bioconjugate Chem.; 1994; 5; 382) are, for example, DOTMA (U.S. Pat. No. 4,946,787), DOTAP (Leventis et al.; Biochim. Biophys. Acta; 1990; 1023; 124), DOGS (EP 394111), DOSPA (WO 9405624), DOSPER (WO 97002419), DMRIE (U.S. Pat. No. 5,264,618) and DC-Chol (Huang et al; Biochem. Biophys. Res. Commun.; 1991; 179; 280; WO 9640067). Those or similar lipids are formulated as such or in combination with so-called co-lipids (for example DOPE) generally in ethanolic, aqueous buffer solutions in the form of micelles or liposomes. They are obtainable, as such or in the form of an oil or solid substance for self-formulation, as commercially available reagents, such as Lipofectin, Lipofectamin, Lipofectamine 2000 (Invitrogen), Fugene (Roche), Effectene (Qiagen), Transfectam (Promega), Metafectene (Biontex) etc.

In the presence of DNA or RNA, cationic lipids and cationic polymers spontaneously form so-called lipoplexes or polyplexes as a result of the opposite charge relationships. DNA is condensed by the compensation of the negative charge on the phosphate radical, that is to say is minimised in size. In general, the transfection efficiency of lipoplexes or polyplexes is dependent upon a large number of parameters. The most important are the relative proportion of genetic material to cationic component in the preparation of the lipo/polyplexes, ionic strength during the preparation of the lipo/polyplexes, absolute quantity of lipo/polyplexes per cell, cell type, proliferation state of the cells, physiological state of the cells, cell division rate, incubation time, etc. Those influencing parameters are the expression of a complicated transfection event in which the lipo/polyplexes or the genetic materials they contain have to overcome a large number of cellular barriers.

The first barrier is the outer negatively charged cell membrane. It is assumed that transfection-active lipoplexes, which need to have a positive net charge, pass into the interior of the cell by adsorptive endocytosis or liquid-phase endocytosis. As a result of the endocytosis, which is an active transport process of the cell, material on the cell surface is surrounded by cell membrane and internalised as a vesicle (endosome). By fusion with so-called lysosomes, which contain a complex mixture of enzymes, the substances contained in the endosomes are degraded. Because a low pH value is necessary for that degradation, endosomes have proton pumps which pump protons into the endosomes until a suitable pH value is obtained. In order to ensure charge neutrality, chloride ions flow into the endosomes to the same extent.

For that reason, many modern cationic lipids or polymers have buffer properties. In that way, the low pH value is not achieved and an influx of ions into the endosomes occurs which causes the endosomes to rupture as a result of the osmotic pressure that develops. In that way, those lipo/polyplexes pass into the cytosol. Since a number of transfection-active lipids and polymers without buffer properties are also known, there must be a further mechanism which allows the lipo/polyplexes to pass into the cytosol. It is supposed, at least in the case of lipids, that there is fusion of the membranes involved and, associated therewith, destabilisation. It is unclear whether, in that process, predominantly the lipoplex or the contained DNA/RNA itself passes into the cytosol. It is supposed, however, that the DNA is released from the lipoplex in the cytosol, because attempts to achieve protein expression by microinjection of lipoplexes directly into the cell nucleus have failed. It would appear that the DNA bound in the lipoplexes is not accessible to the transcription apparatus.

If siRNA or antisense molecules directed against mRNA are involved, the biological site of action is reached and the duration of the action depends substantially upon the concentration of cytosolic RNases and the rate of release from the lipo/polyplexes. DNA cannot per se penetrate the cell nucleus, which is referred to as the “nuclear barrier”. It does, however, pass to its site of action during cell division and thus results in expression of proteins.

As further non-viral methods based on chemical methods there may be mentioned systems which carry a DNA-binding molecule part and a ligand which is capable of triggering receptor-mediated endocytosis (Example transfer infection; Wagner et al.; Proc. Natl. Acad. Sci.; 1990; 87; 3410).

Other compounds consist of a DNA- and/or RNA-binding domain and a ligand which is able to trigger a membrane transfer; for example Penetratin, Derossi et al.; Trends in Cell Biology; 1998; 8; 84 or HIV Tat Petid, Gratton et al.; Nature Medicine; 2003; 9(3); 357. Membrane transfer is to be understood as meaning that a molecule can pass from one side of the membrane to the other side. As DNA- and/or RNA-binding domain there come into consideration all structural elements of a compound which are able to bind DNA and/or RNA by way of electrostatic interactions (for example cations) or hydrogen bridge bonds (for example peptide nucleic acids, PNAs). It is also possible to use intercalating compounds (for example acridine) for binding RNA/DNA. The compounds able to trigger receptor-mediated endocytosis or membrane transfer can also be covalently bound to the genetic material, however, provided the biological action is not impaired or is impaired only slightly thereby.

The most significant example of a non-viral method based on a physical process is electroporation. In that process, the cells to be transfected are introduced between two electrodes to which a customary voltage gradient is applied. In that way, the cells are subjected to an intense electrical current surge (pulse) which results in reversible opening (pores) of the cell membrane. As a result of those pores, substances, such as, for example, genetic material located in the immediate vicinity of the pores are able to penetrate the cell. The pulse (that is to say voltage gradient), being one of the most important parameters of success, must be optimised for each cell type. A number of electroporators have since become available from commercial suppliers (for example Eppendorf/Multiporator, U.S. Pat. No. 6,008,038, Biorad/Gene pulser, U.S. Pat. No. 4,750,100, Genetronics Inc., U.S. Pat. No. 5,869,326, BTX/ECM series) which have been developed specifically for eukaryotic cells and allow matching of the pulse parameters to the cell type in question. In fact, devices are also now available which make in vivo application possible. In the case of in vitro application, the cells are suspended in an electroporation buffer, are introduced, together with the DNA/RNA to be transfected, into an electroporation cuvette provided with electrodes and are subjected to one or more pulses. In addition to the voltage gradient, further important parameters are the nature of the buffer, the temperature, the cell concentration and the DNA concentration. After the cells have been subjected to the pulse, they are left for a short time for regeneration of the cell membrane. The cells are then sown in a culture vessel and cultured in the usual way.

As further physical methods there may be mentioned microinjection, hydrodynamic methods, ballistic methods (gene gun) or methods using ultrasound, as well as the injection of naked DNA into different organs, which results in very little expression of the genes in question.

Processes that combine physical methods and chemical methods also include, in particular, magnetofection which uses DNA-binding molecules on magnetic nanoparticles in order, by means of a magnetic field gradient, to increase the concentration of DNA also of the surface of cells and to trigger endocytosis.

The enormous opportunities afforded by the introduction of genetic material into eukaryotic cells are set against an arsenal of methods having only unsatisfactory efficiency. The shortcomings that specifically arise in the case of each of the methods existing to date relate essentially to the important parameters of efficiency, toxicity, immunogenicity, targeting, restriction in respect of the size of the genetic material, the scope for in vivo/in vitro application, the scope for high throughput applications, the hazard potential, the simplicity of the method and the costs of the method. No method is able to meet all of those parameters to a sufficient extent. The fact that, despite considerable research efforts, it has not been possible hitherto to establish any medical treatment based on gene therapy is attributable to the lack of a suitable gene carrier system.

In particular, the innate immune system of eukaryotes can represent a considerable barrier to non-viral gene delivery systems. The reason for this is that the innate immune system of eukaryotes is able to recognise foreign genetic material by means of Toll-like receptors and to initiate signal transduction cascades that trigger an antiviral state of cell populations. Such an antiviral state of a cell also represents a barrier to transfection with a non-viral gene delivery system, which is impossible or extremely difficult to overcome.

For example, repetitive lipofection experiments, in which a transfection with an siRNA directed specifically against a certain protein was carried out first of all and was then followed by a plasmid transfection with a reporter gene, showed that successful transfection of the plasmid did not occur, although the cells appeared healthy. Only in the case of very small amounts of siRNA was it possible to detect a small amount of the reporter protein.

In order to rule out the possibility of its being an off-target effect of the specific siRNA, the experiment was repeated with a non-specific siRNA which was “blasted” towards the human genetic material. The result remained the same, however.

Because the proliferation of cells is known to have an effect also in the case of lipofection, an investigation was carried out as to whether the proliferation behaviour of the cells had been impaired by the pre-transfection with siRNA. That investigation established that proliferation rates fall in the case of relatively large amounts of siRNA, but sufficient proliferation was achieved in the experiments when the plasmid transfection following the pre-transfection failed. It appeared, surprisingly, as if the cells were able to protect themselves against the second transfection.

Using repetitive transfection experiments in which two plasmid transfections were carried out one after the other, a similar result was obtained, although not with the clarity mentioned above. The second transfection step was frequently either very inefficient or counterproductive. In this case too, investigations similar to those mentioned above were carried out in order to ensure that toxic effects were not involved. Further investigations showed that interferons were secreted.

Transfection of siRNA for Triggering RNA-Interference

Genes can be switched off selectively by the introduction of dsRNA into cells when the mRNA has sequence homology with the inserted dsRNA.

The process is referred to as RNA-interference (Fire et al., Nature, 1998, 391, 806-811) and usually proceeds as follows: a dsRNA, inserted into the cell, having a homologous sequence of an mRNA inherent in the cell is cut by the dicer enzyme into a large number of small dsRNA fragments of 21 to 25 nucleotides (Bernstein et al., Nature, 2001, 409, 363-366).

Dicer is an ATP-dependent ribonuclease. The resulting nucleotide fragments have 2-3 nucleotides overhanging at the 3′-end. The small RNA pieces are referred to as “small interfering RNA” (siRNA) (Elbashir et al., Nature, 2001, 411, 494-498).

The double-stranded siRNAs are unwound, with consumption of ATP, presumably with the aid of a helicase (Dalmay et al., EMBO, 2001, J20, 2069-2078). A single strand is then converted into the protein complex RISC (RNA-induced silencing complex) (Kuhlmann et al., Biol. unserer Zeit, 2004, 3, 142-150). The strand remaining on the RISC can be hybridised with a complementary RNA (target RNA). The target-RNA is then cut by an integral endoribonuclease. Because genes are able to act only by the circuitous route of single-stranded mRNA, that gene is thus de facto switched off. Although it is still transcribed, the RNA-interference (RNAi) degrades that mRNA again just as quickly. For that reason, the RNA-interference is also referred to as “Post-Transcriptional Gene Silencing” (PTGS).

RNA-interference is found in protozoa, fungi, plants and animals, although the individual mechanisms differ slightly from one another. Archaebacteria and prokaryotes do not have that ability.

The question of function has not yet been precisely clarified. It is assumed to serve for protection against RNA viruses. For example, plants infected with viruses are able to recover and in the case of newly developed leaves the symptoms decline. Symptom-free leaves can no longer become infected with viruses of a related kind. Complementary copies of the invading viruses or their RNA are created which serve as a matrix for the synthesis of the original RNA. A virus-specific dsRNA is formed which triggers the PTGS mechanism. Since, initially, the concentration of dsRNA is too low, the plant is able to recover and gain control over the viruses only gradually. It is assumed that an RNA-dependent RNA-polymerase (RdRP) inherent in the cell recognises the single-stranded virus genome, converts it into dsRNA and then starts the RNAi process. The theory that PTGS protects against viruses has been supported by the finding of inhibitors of PTGS in viruses. It is still not known exactly how they work and whether plants have in turn developed mechanisms against those inhibitors.

RNA-interference has gained immense importance in recent years, because it offers the possibility of switching off undesirable genes or proteins and thus of controlling viral diseases and other diseases. Furthermore, it has also become an indispensable aid in research aimed at uncovering gene-function relationships.

The most commonly employed variant of using RNA-interference lies in the transfection of synthetically produced siRNA molecules, that is to say double-stranded RNA having a 3′-overhang of 2-3 nucleotides. In mammalian cells, an inserted dsRNA having more than 30 by brings about enzymatic destruction of all mRNAs and stops protein synthesis (Kaufmann, Proc. Natl. Acad. Sci. USA, 1999, 96, 11693-11695). After the injection of longer dsRNA, some higher eukaryotes can react with the production of interferons, which can inhibit the expression of viral genes and steer the cell into apoptosis. If that is to be prevented, in the case of mammalian cells the length of the siRNA must be less than 30 by (Tuschl et al., Genes Dev., 2001, 15, 188-200). As transfection methods, the methods known from DNA transfection are available. The difference with respect to DNA transfection lies solely in the site of action of the inserted genetic material. In the case of DNA transfection, that site is the nucleus. In the case of siRNA transfection it is the cytosol.

The most commonly employed methods are electroporation, transfection by cationic polymers and, especially, transfection by cationic lipids. The best reagents for plasmid transfection are not necessarily also the best reagents for siRNA transfection and vice versa. Special reagents suitable for siRNA transfection are therefore commercially available. Examples are Interferrin (Polyplus), X-treme Gene siRNA (Roche), siPort (Ambion), Silentfect (Biorad), Dharmafect (Dharmacon) and Lipofectamin RNAiMax (Invitrogen).

A measure of the success of transfection, in the case of siRNA transfection, is understood as being the relative knock-down of a protein or of a gene as compared with an untreated sample or a sample transfected with non-specific siRNA (siRNA without a target). A problem in the case of siRNA transfection is so-called off-target effects, which are to be understood as being, for example, the unintentional impairment of the expression of a gene that is not the target of the knock-down. That can occur, for example, in the case of sequence similarities. In order to keep such off-target effects as low as possible and for reasons of toxicity, it is a requirement of potential siRNA transfection systems that they achieve the highest possible knock-down using the smallest possible amount of siRNA. A further preferred requirement, especially in the case of in vivo applications, is that, apart from the expression of the target protein, the expression profile of the cells be changed as little as possible.

### BRIEF DESCRIPTION OF THE FIGURES

FIG. 1 shows a comparison of transfection results of HeLa cells after simple and repetitive transfection with various amounts of sheep polyclonal antibody against human IFNβ.

FIG. 2 shows a comparison of transfection results of HeLa cells after simple and repetitive transfection with various amounts of mouse monoclonal antibody against human interferon alpha/beta receptor chain 2 (CD118).

FIG. 3 shows a comparison of transfection results of HeLa cells after simple and repetitive transfection with various amounts of mouse anti-human-CD282 antibody (=anti-TLR2).

FIG. 4 shows a comparison of transfection results of HeLa cells after simple and repetitive transfection with various amounts of mouse anti-human TLR3 antibody.

FIG. 5 shows a comparison of transfection results of HeLa cells after simple and repetitive transfection with various amounts of mouse anti-human-CD284 antibody (=anti TLR4).

FIG. 6 shows a comparison of transfection results of HeLa cells after simple and repetitive transfection with various amounts of mouse anti-human TLR1 antibody.

FIG. 7 shows a comparison of transfection results of HeLa cells after simple and repetitive transfection with various amounts of human interleukin-1 receptor antagonist (IL1-ra human).

FIG. 8 shows a comparison of transfection results of HeLa cells after simple and repetitive transfection with various amounts of kinase inhibitor U0126, i.e. 1,4-diamino-2,3-dicyano-1,4-bis(o-aminophenylmercapto)butadiene.

FIG. 9 shows a comparison of transfection results of HepG2 cells after simple and repetitive transfection with various amounts of mouse anti-human-CD282 antibody (=anti-TLR2).

FIG. 10 shows a comparison of transfection results of HeLa cells after simple and repetitive transfection with various amounts of rabbit anti-human TLR9 antibody.

FIG. 11 shows siRNA transfection results of HeLa-Luc cells in the presence of MEK kinase inhibitor U0126.

FIG. 12 shows siRNA transfection results of cells in the presence of TLR7/8 agonists imiquimod and ssRNA40/LyoVec, respectively.

### DESCRIPTION OF THE INVENTION

The problem of the invention is to provide a method that allows more efficient transfection. That applies both to single transfection and to repeated transfection, that is to say transfection two or more times. A further problem is to affect the physiological state of the cell population as little as possible, that is to say that the protein expression profile of the cell population should ideally be changed only in respect of the proteins the genes of which have been inserted into the cell or the expression of which is reduced or blocked by the inserted genetic material. In the case of transfection of siRNA, however, it can also be advantageous to change the physiological state of the cell intentionally in order to bring the cells into an “antiviral state”, because in that case the RNA-interference proceeds especially efficiently. Furthermore, a composition and a kit of parts is provided which contain the components for carrying out more efficient transfection of eukaryotic cells with non-viral gene delivery systems.

That problem is solved according to the invention by a method for improving the transfection result of non-viral gene delivery systems, characterised in that

a) the cells are treated before and/or during transfection with at least one means for at least partially suppressing the innate intracellular and/or intercellular immunity and, during transfection, genetic material, especially modified and/or unmodified ssDNA, modified and/or unmodified dsDNA, modified and/or unmodified ssRNA, modified and/or unmodified dsRNA and/or modified and/or unmodified siRNA, is introduced into the cells; or

b) the cells are treated before and/or during and/or after transfection with at least one means for at least partially activating the innate intracellular and/or intercellular immunity and, during transfection, modified and/or unmodified siRNA is introduced into the cells.

The method according to the invention for improving the transfection result can be carried out in vitro and/or in vivo.

By virtue of the at least partial suppression of the innate intracellular and/or intercellular immunity, that is to say one of the intracellular and/or intercellular signal transduction cascades of the innate immunity is interrupted, the transfection result can be improved and/or undesirable changes in the expression profile of a transfected cell can be avoided.

That is to say, that especially the intracellular signal transduction cascade starting from the TLRs, via the adapter molecules, via the corresponding kinases, which in turn induce cytokines, especially the interferons, via the activation of transcription factors, and membrane transport processes can be down-regulated, interrupted or weakened. Furthermore, signal transmission by messenger substances between the cells can be interrupted. Since they are all proteins, according to the invention it is preferable for activity-increasing or activity-reducing effectors such as antibodies, aptamers, antagonists or inhibitors of those proteins to be used. The corresponding active substances can be introduced to or into the cells as such or using suitable auxiliary molecules, depending upon cell permeability or the target site. For example, active substances can be introduced into the endosomes by means of liposomal carriers. If the target site is the cytosol, possible methods include especially electroporation or specific peptide sequences capable of rendering the cell walls permeable. If the active substances are peptides or proteins, according to the invention they can be formed intracellularly also by transfection of suitable genetic material and, if necessary, directed to the corresponding cell compartments using localisation sequences. If knock-down is to be effected by means of siRNA genes that code for crucial proteins of the signal transduction apparatus, according to the invention the known transfection systems are available.

The method according to the invention can be used and the use according to the invention can be effected both in vitro and in vivo. It can be used for the purpose of preventing the development of the “antiviral state” of cells during transfection or even beforehand. Since the cells, during culturing, can also come into contact with biological material, for example serum or trypsin, which may contain substances to which one or more TLR respond (for example DNA, RNA, LPS etc.), it can happen that cells are already in an antiviral state before the transfection is begun. Given that background it also becomes understandable why the reproduction of transfection results is considered difficult. The quality of transfection results is highly dependent upon the immunological starting state of the cells which, in turn, depends upon the pre-treatment (for example sub-culturing).

In the method according to the invention, the non-viral gene delivery system can comprise a cationic lipid, a cationic polymer or a cationic protein; and/or can comprise a compound which has a DNA- and/or RNA-binding domain and is able to trigger receptor-mediated endocytosis or a membrane transfer; and/or can comprise a compound which is covalently bound to DNA and/or RNA and is able to trigger receptor-mediated endocytosis or a membrane transfer; and/or can be based on a physical method such as electroporation, microinjection, magnetofection, ultrasound or a ballistic or hydrodynamic method.

Furthermore, in the method according to the invention the transfection can be carried out at least twice, that is to say two or more times (3, 4, 5, 6, etc.).

Moreover, in the method according to the invention, preferably a cationic lipid can be contained in the non-viral gene delivery system, especially a cationic lipid according to formula (I):

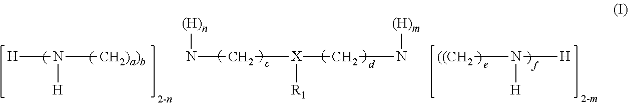

wherein

R₁ may be

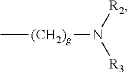

wherein

R₂ and R₃ each independently of the other may be dodecyl, dodecenyl, tetradecyl, tetradecenyl, hexadecyl, hexadecenyl, octadecyl, octadecenyl or other alkyl radicals which, in all possible combinations, may be saturated, unsaturated, branched, unbranched, fluorinated or non-fluorinated and may be composed of from 5 to 30 carbon atoms;

X may be

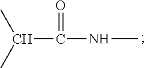

and

wherein m=0 and n=0; or m=0 and n=1; or m=0 and n=2; or m=1 and n=1; or m=1 and n=2; or m=2 and n=2; and

g may be 1, 2, 3, 4, 5, 6, 7 or 8; a may be 0, 1, 2, 3, 4, 5 or 6; b may be 0, 1, 2, 3, 4, 5 or 6; c may be 0, 1, 2, 3, 4, 5 or 6; d may be 0, 1, 2, 3, 4, 5 or 6; e may be 0, 1, 2, 3, 4, 5 or 6, and f may be 0, 1, 2, 3, 4, 5 or 6.

More preferably, in the method according to the invention, a non-viral gene delivery system comprising a cationic lipid according to formula (I) can be used, wherein R₂ and R₃ each independently of the other may be dodecyl, dodecenyl, tetradecyl, tetradecenyl, hexadecyl, hexadecenyl, octadecyl, octadecenyl; m and n may be 1; and g may be 1, 2, 3, 4, 5, 6, 7 or 8; a may be 0, 1, 2, 3, 4, 5 or 6; b may be 0, 1, 2, 3, 4, 5 or 6; c may be 0, 1, 2, 3, 4, 5 or 6; d may be 0, 1, 2, 3, 4, 5 or 6; e may be 0, 1, 2, 3, 4, 5 or 6, and f may be 0, 1, 2, 3, 4, 5 or 6.

In the method according to the invention, the innate intracellular and/or intercellular immunity can be at least partially suppressed by at least one antibody, intrabody, aptamer, antagonist, inhibitor and/or an siRNA, which block(s) the transmission of an intracellular and/or intercellular signal of the innate immunity. Corresponding antibodies, intrabodies, aptamers, antagonists and inhibitors are commercially available.

Furthermore, in the method according to the invention it is possible by means of knock-down with siRNA to switch off at least one gene that codes for a protein necessary for signal transduction. Corresponding siRNA or plasmids, shRNA (short hairpin RNA), for example directed against TLRs, kinases and transcription factors, are to some extent commercially available (Imgenex/Invivogen).

In the method according to the invention, for at least partial suppression of the innate intracellular and/or intercellular immunity it is possible for at least one of the group TLR 1, TLR 2, TLR 3, TLR 4, TLR 5, TLR 6, TLR 7, TLR 8, TLR 9, TLR 10, TLR 11, TLR 12, TLR 13, CD14, CD38, RIG-I helicase and/or RIG-I-like helicase to be blocked. It is especially preferable to block TLR 1, TLR 2, TLR 4 and/or TLR 9. It is further preferable to block a plurality of the above-mentioned receptors or proteins. According to the invention, suitable antibodies are those capable of blocking the TL receptors. For example, antibodies to the Toll-like receptors TLR1, TLR2, TLR3, TLR4, TLR5, TLR6, TLR7, TLR8, TLR9 and CD14 are known and are commercially available. Examples of antibodies capable of blocking human TLR are: mouse anti-human-CD282 antibody (=anti TLR2), monoclonal, AbD Serotec, Cat. No.: MCA2484EL, mouse anti-human TLR3 antibody, monoclonal, Lifespan Biosciences Cat. No.: LS-C18685, mouse anti-human-CD284 antibody (=anti TLR4), monoclonal, AbD Serotec, Cat. No.: MCA2061 EL, antibody: mouse anti-human TLR1 antibody (monoclonal, 0.05% sodium azide, 100 μg lyophilisate); Invivogen, No. Mab-htlr1, rabbit anti-human-TLR9 antibody (=anti TLR9), polyclonal, 0.5 μg/μl in PBS, 0.2% gelatin, 0.05% sodium azide, Lifespan Biosciences, Cat. No.: MCA2484EL. If undesirable substances, such as sodium azide, are contained in the commercially available antibodies, those undesirable substances must be removed, for example by dialysis, before the antibody is used in the method according to the invention.

Antibodies capable of blocking TLR can be inserted into the endosomes together with endocytosis-triggering lipoplexes or polyplexes, as such or packed in liposomes, and thus block the receptors. Generally, however, addition to the extracellular space is also sufficient. According to the invention, the choice of the receptor to be blocked of course also depends upon the nature of the genetic material to be transfected. Furthermore, the transfecting agent can also determine the choice of the receptor to be blocked. The TLR that detect the genetic material, that is to say TLR 3, TLR 7, TLR 8 and TLR 9, are usually located in the endosomes. The other TLR are located on the plasma membrane. If, for example, DNA is to be transfected, preferably TLR 9 can be blocked. If the DNA is of bacterial origin, it is preferable to block TLR 4 and/or TLR 5 in addition to TLR 9.

In a preferred embodiment, an antibody concentration of from 0.01 to 100 μg/ml is used by addition to the culture medium. In a further preferred embodiment, the concentration of the antibody is from 0.01 to 10 μg/ml culture medium and in the most preferred embodiment from 0.01 to 5 μg/ml.

The antibody can also be integrated into the transfection-active complex, for example lipoplex. In a preferred embodiment, the ratio antibody:genetic material is from 0.01:1 (μg/μg) to 10:1 (μg/μg). In a more preferred embodiment, the amount is from 0.01 to 1 μg/μg and in the most preferred embodiment from 0.01 to 0.3 μg/μg of genetic material.

The following remarks apply to all antibodies that can be used according to the invention as means for at least partially suppressing the innate intracellular and/or intercellular immunity: the antibody/antibodies used must generally be directed against the target molecule to be blocked, for example a receptor such as a TLR, cytokine receptor, interferon receptor, etc. of the cells that are to be transfected. If, for example, a TLR of a human cell is involved, the antibody must generally be directed against the human TLR receptor that is to be blocked. In many cases, the antibodies are also cross-reactive on account of the great similarity between the target molecules of various species, for example TLRs; that is to say, although an antibody to a target molecule of one species has been developed, it also exhibits its properties against a similar target molecule of another species. Also preferred is the use of antibodies from cells of the same species that is to be transfected, because in that case they exhibit little or no immunogenicity. The antibodies may also have been prepared recombinantly. If they are to be used on human cells, they can be “humanised”. Preferably, the antibody used binds to its target molecule with a high degree of affinity. The antibody used can be polyclonal or monoclonal, with preference being given to monoclonal antibodies. It is also possible to use a modified antibody, for example in the case of a modified antibody the Fc fragment can be absent, because the binding to the target molecule/antigen is brought about solely by the Fab fragments. A modified antibody can also have been modified with hydrophobic groups, such as, for example, lipids, in order to facilitate the anchoring thereof in membranes and/or lipsomes. If it is desirable to detect the antibody used more easily, it can also be labelled with a fluorescent dye. It is also possible to carry out a plurality of modifications on an antibody. Furthermore, the antibody used must be free of additives that prohibit use on living cells, such as, for example, certain preservatives.

In the method according to the invention it is also possible for at least one of the kinases IRF kinase, TBK1, MAP kinase, MAPK kinase, MAPK kinase, MAPKK kinase, MAPKKK kinase, MEK1, MEK2, MEK5, MKK4/SEK, MKK5, MKK6, MKK7, ERK1, ERK2, ERK3, ERK4, ERK5, ERK6, ERK7, ERK8, JAK, JNK1, JNK2, p38 MAP kinase, RK, p38/RK MAP kinase, p30/RK MAP kinase, phosphatidyl inositol 3-kinase, IRAK-1, IRAK-4, IKK-alpha, IKK-beta, IKK gamma, IKK delta, IKK epsilon, TAK1, PKB kinase, PKD1, PKD2, MSK1 or PKR to be blocked. Preferably at least one kinase selected from MEK1 and MEK2 is blocked. It is further preferable for a plurality of the above-mentioned kinases to be blocked.

Furthermore, according to the invention the kinase MEK1 and/or MEK2 can be inhibited by a compound having an 1050 value of less than 100 nM. Moreover, according to the invention the kinase MEK1 and/or MEK2 can be blocked by 1,4-diamino-2,3-dicyano-1,4-bis(o-aminophenylmercapto)butadiene (U0126). In a preferred embodiment, a concentration of from 1 to 500 μM is used. In a more preferred embodiment, the concentration is from 1 to 100 μM and in the most preferred embodiment from 1 to 30 μM.

In the method according to the invention it is also possible to block at least one cytokine, at least one tumour necrosis factor (TNF), at least one interleukin and/or at least one interferon that are involved in the innate intercellular immunity.

As the interferon to be blocked there comes into consideration, for example, an interferon of type I, especially at least one of the interferons selected from interferon-alpha, especially interferon-beta, interferon-gamma and interferon-omega. As the interleukin to be blocked, especially interleukin-1 comes into consideration.

It is also possible for at least one receptor for cytokines to be blocked, especially at least one receptor for interferons, especially of type I, at least one receptor for interleukins, and/or at least one receptor for tumour necrosis factors. An example of a suitable antibody directed against the interferon-typed-receptor is the Mouse monoclonal Antibody against Human Interferon Alpha/Beta Receptor Chain 2 (CD118), clone MMHAR-2, isotype Ig2a, C=0.5 mg/ml in PBS (phosphate buffered saline) containing 0.1% bovine serum albumin (BSA), PBL Biomedical Laboratories, Product No. 21385. In particular, the receptor for interleukin-1 can be blocked by an antibody or an antagonist such as IL-ra (Human Interleukin-1 Receptor Antagonist, Biomol. Cat. No.: 54592). A further preferred target is the interferon-gamma-receptor.

The following remarks apply to all antagonists that can be used according to the invention as means for at least partially suppressing the innate intracellular and/or intercellular immunity: the antagonist(s) used must generally be directed against the target molecule to be blocked, for example a receptor such as an interleukin receptor, cytokine receptor, interferon receptor, etc. of the cells that are to be transfected. If, for example, a receptor of a human cell is involved, the antagonist must generally be directed against the human receptor that is to be blocked. In many cases, the antagonists are also cross-reactive on account of the great similarity between the target molecules of various species, for example TLRs; that is to say, although an antagonist against a target molecule of one species has been developed, it also exhibits its properties against a similar target molecule of another species. Also preferred is the use of antagonists from cells of the same species that is to be transfected, because in that case they exhibit little or no immunogenicity. The antagonists may also have been prepared recombinantly or synthetically. Preferably, the antagonist used binds to its target molecule with a high degree of affinity. Antagonists used according to the invention can also have been modified analogously to the modified antibodies.

In a preferred arrangement of the invention, a plurality of the above-mentioned receptors and/or proteins involved in the signal transduction cascade for triggering the innate intracellular and/or intercellular immunity are blocked. For example, a plurality of antibodies to TLR receptors can be combined in order to utilise additive effects and/or synergistic effects. Equally, for example, inhibitors and/or antibodies, and/or intrabodies, and/or aptamers, and/or antagonists, and/or siRNA against TLR receptors and/or TLR assisting proteins and/or adapter molecules and/or kinases and/or transcription factors and/or cytokines and/or cytokine receptors can also be combined in order at least partially to interrupt the signal transmission cascade of the innate immunity.

The method according to the invention can also be used for improving the transfection results of siRNA transfections with non-viral gene delivery systems in several ways, the siRNA being modified or unmodified.

In the method according to the invention, an agonist can be used as means for activating the innate immunity.

Firstly, the method according to the invention can be used for activating the RNA-interference machinery by stimulation of the intracellular part of the innate immune system. The activation is effected by loading various TLR receptors with corresponding agonists, but especially by loading (and thus activating) the receptors TLR7 and TLR8. The receptors TLR7 and TLR8 are located in the endosomes and detect ssRNA, as is to be expected in the case of an infection of a RNA virus. The activation of those receptors results in an especially high “antiviral state” to which an active RNA-interference machinery can be allocated. The assumption that RNA-interference is a mechanism for protection against viruses fits easily into the overall picture. The improvement in the transfection results of siRNA transfections differs from transfections with other genetic material in the respect that for a good transfection result there needs to be available an active RNAi machinery which is part of the innate immune system, the active RNAi machinery overcompensating for the adverse effect of the innate immune system on the uptake of the siRNA.

Preferably, in the method according to the invention the innate intracellular and/or intercellular immunity can be at least partially activated by at least one agonist for a TL receptor, especially by at least one agonist for TLR7 and/or TLR8.

According to the invention, the at least one agonist for TLR7 and/or TLR8 can be selected from the group comprising bropirimine (2-amino-5-bromo-6-phenyl-4-pyrimidinone), imidazoquinolines, thiazoloquinolines, guanosine analogues and ssRNA.

Preferably, in the method according to the invention the at least one agonist can be imiquimod (R837, 1-(2-methylpropyl)-1H-imidazo[4,5-c]quinoline-4-amine), resiquimod (R848, 4-amino-2-(ethoxymethyl)-a,a-dimethyl-1H-imidazo[4,5-c]quinoline-1-ethanol) or gardiquimod (1-(4-amino-2-ethylaminomethylimidazo[4,5-c]quinolin-1-yl)-2-methylpropan-2-ol); or CL075 or CL097; or loxoribine (7-allyl-7,8-dihydro-8-oxo-guanosine) or isatoribine (7-thia-8-oxoguanosine); or ssRNA having U-rich and/or GU-rich sequences, especially ssRNA having the sequence motifs UGUGU and/or GUCCUUCAA. Imiquimod can be used in a concentration of from 0.1 to 100 μg/ml, preferably in a concentration of from 0.1 to 20 μg/ml, and most preferably in a concentration of from 0.1 to 10 μg/ml. The addition is preferably made at the same time as the transfection and/or at a later stage. Preferably, the ssRNA has a length of at least 15 bases. Furthermore, the ssRNA can have a phosphothioate backbone. For example, an ssRNA having a length of 20 nucleotides can be used in a concentration of from 0.1 to 100 μg/ml, preferably from 0.1 to 20 μg/ml and most preferably from 0.1 to 10 μg/ml. The addition of the ssRNA is preferably made at the same time as the transfection and/or after the transfection. Preferably, the immunostimulatory ssRNA can be contained in the transfection-active complex comprising the non-viral gene delivery system and siRNA or plasmid-DNA on which shRNA has been encoded.

The cell-permeable agonists can be added directly to the medium of the cells before, during or after the actual siRNA transfection step, it being necessary for the ssRNA and the analogues thereof to be complexed with suitable transfection reagents, because they are not inherently cell-permeable and thus do not reach the TLRs located in the endosomes. It is also possible to incorporate the agonists into lipoplexes or polyplexes together with the siRNA or, by derivatisation with alkyl chains, to incorporate the agonists into the lipid membranes of the liposomes or lipoplexes which consist of the actual transfection-active cationic lipids and possible co-lipids such as, for example, DOPE. The agonists can also be covalently bound to cationic polymers.

Secondly, the method according to the invention can be used for activating the RNA-interference machinery by stimulation of the intercellular part of the innate immune system. The activation is effected by loading receptors of antiviral cytokines with suitable agonists. In a preferred arrangement, interferon-beta and/or interferon-gamma are used in a concentration of from 1 to 10 000 U/ml. In a more preferred embodiment, the concentration is from 1 to 5000 U/ml and in the most preferred embodiment from 1 to 2000 U/ml. The addition is preferably made at the same time as the transfection and/or at a later stage.

On the other hand, the method according to the invention can be used to stimulate parts of the adaptive immune system that are suitable for activation of the siRNA machinery and at the same time to block other parts that influence the amount of siRNA introduced, for example by down-regulation of the endocytosis, it being possible for synergistic effects to be utilised.

In the method according to the invention, the cells can be treated up to 4 days, preferably up to 18 hours, especially up to 6 hours, before transfection with the at least one means for at least partially activating the innate intracellular and/or intercellular immunity.

Furthermore, in the method according to the invention the cells can be treated up to 2 days, preferably up to 12 hours, especially up to 6 hours, after transfection with the at least one means for at least partially activating the innate intracellular and/or intercellular immunity.

Moreover, in the method according to the invention the cells can simultaneously be treated with the at least one means for at least partially suppressing or activating the innate intracellular and/or intercellular immunity and brought into contact with the non-viral gene delivery system.

The method according to the invention can also be used to prevent an undesirable reaction of the innate immune system and thus a modified gene expression of the cell. That is of particular significance in in vivo applications and in the clarification of protein functions by siRNA transfections in complex signal pathways. Different signal transduction pathways of the cell are frequently also coupled with the signal transduction pathways of the innate immune system. In the case of a knock-down of a protein, which simultaneously has a massive influence on the physiological state of the cell, the allocation of the function to the protein is rendered more difficult. In order that this can be avoided, the method according to the invention can be used for partially or completely blocking the response of the innate immune system.

The method according to the invention is suitable for transfection of eukaryotic cells. If the eukaryotic cells are transfected in vitro, the cells can be present adherently or in suspension in a suitable culture medium. Preferably, at the time of transfection the cells are in the logarithmic phase of proliferation if transfection with DNA is involved. If RNA is to be transfected, at the time of transfection the cells are preferably in the lag or log phase. If a transfection is being carried out with the aim of expressing a protein, there comes into consideration as genetic material dsDNA having a component expressible as RNA or as peptide/protein or ssRNA having a component expressible as peptide/protein (in each case modified or unmodified).

If a transfection is being carried out with the aim of achieving a knock-down of a gene by RNA-interference, it is possible to use modified or unmodified dsDNA having a component expressible as small-hairpin-RNA (shRNA) or modified or unmodified siRNA; only in the latter case is activation of the TLR 7 and/or TLR 8 advisable. Additional blocking of TLR and/or cytokine receptors and/or interruption of a signal transduction by blocking of MEK1 and/or MEK2 can be advantageous, however, especially in order as far as possible not to affect the expression profile of the cells.

A method according to the invention can preferably have the following method steps:

(a) provision of a first solution containing an antibody, antagonist, inhibitor or agonist against a target molecule; provision of a second solution containing a non-viral gene delivery system; and provision of a third solution containing the genetic material to be transfected;

(b) addition of the first solution, containing an antibody, antagonist or inhibitor against the target molecule, to the cells to be transfected in the culture medium;

(c) mixing of the second solution containing the non-viral gene delivery system and the third solution containing the genetic material to be transfected;

(d) incubation of the mixture from step (c);

(e) addition of the incubated mixture from step (d) to the cells to be transfected, pretreated with the first solution, from step (b).

According to the invention, the first solution can contain an antagonist or antibody to a cytokine receptor or TLR 1, TLR 2, TLR 3, TLR 4, TLR 5, TLR 6, TLR 7, TLR 8 or TLR 9 as target molecule; an inhibitor for the target molecule MEK1 and/or MEK2; or an agonist for TLR 7 and/or TLR 8; especially one of those mentioned above. An antagonist or antibody can preferably be dissolved in a buffered salt solution, for example PBS, basal medium, etc., with a physiological pH value of from 6.8 to 7.45 and a physiological osmolality of from 270 to 310 mosmol/kgH₂O, or in water or in unbuffered salt solution having a pH value of from 5 to 9. In the event of solubility problems, the inhibitor can also be diluted in a suitable physiologically acceptable solvent, for example ethanol/DMSO. The concentration of the antagonist, antibody or inhibitor in the first solution can be from 0.01 to 1 μg/μl.

According to the invention, the addition of the first solution to the cells to be transfected in the culture medium in step (b) can be effected during a period of from 24 hours to 1 sec before the treatment of the cells with the transfection complex (step (e)), preferably during a period of from 0.5 to 5 hours. That applies especially when the first solution contains an MEK1- and/or MEK2-inhibitor, or an antibody or antagonist directed against TLR 1, TLR 2, TLR 3, TLR 4, TLR 5 or TLR 6 or cytokine receptors. The addition is made by adding a suitable amount of the first solution to the culture medium of the cells to be transfected. The resulting concentration of the antibody or antagonist in the culture medium of the cells to be transfected can be from 0.01 μg/ml to 50 μg/ml, preferably from 0.05 to 12 μg/ml, especially from 0.1 to 5 μg/ml. The resulting concentration of the inhibitor in the culture medium of the cells to be transfected can be from 1 nM to 500 μM, preferably from 5 to 100 μM, especially from 10 to 50 μM.

In a method according to the invention, the mixing of the second solution containing the non-viral gene delivery system and the third solution containing the genetic material to be transfected (step (c)) can be effected by pipetting. For each cell to be transfected, an amount of genetic material to be transfected of from 0.1 picogram/cell to 300 picogram/cell, preferably from 0.2 to 50 picogram/cell, especially from 1 to 10 picogram/cell, genetic material to be transfected can be used. The amount of the non-viral gene delivery system is governed by the amount of genetic material. In the case where the non-viral gene delivery system is electrostatically bound to the genetic material, the amount is defined by the charge ratio (+/−) between the gene delivery system and the genetic material. The charge ratio (+/−) gene delivery system:genetic material can be from 0.1:1 to 100:1, preferably from 1:1 to 20:1, especially from 4:1 to 10:1. In the case of non-electrostatic binding, the amount of the non-viral gene delivery system is defined by way of the stoichiometric ratio with respect to the genetic material. The stoichiometric ratio non-viral gene delivery system:genetic material can be from 0.1:1 to 1000:1, preferably 1:1.

The mixture of the second solution containing the non-viral gene delivery system and the third solution containing the genetic material to be transfected can be incubated for a period of from 1 min to 30 min, preferably from 10 to 15 min.

Preferably, in the method according to the invention a non-viral gene delivery system can be used which comprises a cationic lipid, especially a cationic lipid according to formula (I) mentioned above.

According to the invention, after 24 hours steps (a) to (e) can be repeated in order to transfect the cells with genetic material a further time. Preferably, prior to a second or further transfection the additive-containing culture medium in which the cells to be transfected are located should be replaced by fresh culture medium.

In a method according to the invention, a first solution containing an antagonist or antibody to TLR 3, TLR 7, TLR 8 or TLR 9 can be mixed with a second solution containing a non-viral gene delivery system and a third solution containing the genetic material to be transfected. Preferably, the first solution containing an antibody/antagonist is added to the second solution containing a non-viral gene delivery system and mixing is carried out. The amount of antibody used is from 0.1% by weight to 50% by weight, based on the amount of the non-viral gene delivery system, preferably from 0.1 to 10% by weight. The amount of the non-viral gene delivery system is governed by the amount of genetic material that is to be used. In the case where the non-viral gene delivery system is electrostatically bound to the genetic material, the amount is defined by the charge ratio (+/−) between the gene delivery system and the genetic material. The charge ratio (+/−) gene delivery system:genetic material can be from 0.1:1 to 100:1, preferably from 1:1 to 20:1, especially from 4:1 to 10:1. In the case of non-electrostatic binding the amount of the non-viral gene delivery system is defined by way of the stoichiometric ratio with respect to the genetic material. The stoichiometric ratio non-viral gene delivery system:genetic material can be from 0.1:1 to 1000:1, preferably 1:1. The mixture of the first and second solutions is preferably incubated for at least 5 min. The third solution containing genetic material to be transfected is then added to the incubated mixture of the first and second solutions and mixing is carried out. For each cell to be transfected, an amount of genetic material to be transfected of from 0.1 picogram/cell to 300 picogram/cell, preferably from 0.2 to 50 picogram/cell, especially from 1 to 10 picogram/cell, genetic material to be transfected can be used, the antibody/antagonist being incorporated into the transfection-active complex comprising non-viral gene delivery system and genetic material. Preference is given to the use of antibodies/antagonists that have been modified with a lipophilic molecule moiety in order to facilitate binding of the antibody/antagonist into the lipoplex. It is also possible to use antibodies that have been linked to the non-viral gene delivery system by way of avidine/streptavidine and biotin. In that case the gene delivery system and the antibody need to be suitably modified beforehand. It is also possible for an antibody directed against TLR 3, TLR 7, TLR 8 and TLR 9 to be linked covalently to the non-viral gene delivery system.

Methods of providing cationic immunoliposomes of liposomes comprising cationic lipids or lipoplexes, that is to say complexes containing DNA and cationic lipids, covalently with antibodies or antibody fragments are known. The methods have been developed in order to enable gene delivery systems to be targeted in the direction of target cells in the body. Most of the methods use so-called cross-linkers. Cross-linkers are bifunctional molecules which create a covalent link between two molecules having corresponding functional groups. For example, Pierce offers a wide selection of water-soluble and water-insoluble heterobifunctional (that is to say suitable for linking two different functional groups) and homobifunctional (suitable for linking two identical functional groups) cross-linkers. Carboxyl groups and amino groups can be used for linking the antibodies; in the case of Fab-fragments it is also possible to use the thiol group for a linkage. Cationic lipids and polymers contain amino functions for the linkage. In the case of cationic peptides, carboxyl and amino groups can be used for the linkage.

Cationic immunoliposomes can be formulated in the same way as conventional liposomes by derivatising antibodies with lipids and adding them to the cationic lipids (possibly with co-lipids) prior to formulation. For covalent linkage by means of homobifunctional cross-linkers, such as, for example, DSP (dithiobis[succinimidylpropionate]) amino functions can be used to link a lipid having an amino function covalently to an antibody. For covalent linkage of a Fab-fragment to a lipid it is also possible to use a thiol function and the cross-linker N-succinimidyl-4-(p-maleimidophenyl)butyrate (Martin et al.; J. Biol. Chem.; 1982 257(1), 286) or SPDP (N-succinimidyl 3-(2-pyridyldithio)-propionate). If the cross-linkers are not soluble in water, those coupling reactions between antibody and lipid must be carried out in organic solvents and purification, that is to say removal of the organic solvent, must take place prior to a transfection. Dialysis can be used for purification. If the cross-linker is soluble in water, for example DSP, a covalent linkage between the lipid and the antibody, for example linkage via amino functions, can be effected in aqueous solution and purification prior to transfection is not absolutely necessary.

Methods for the preparation of such lipoplexes having modified antibodies that are covalently bound to a lipid are known to the person skilled in the art. The lipoplexes then take up the modified antibodies (Lee et al., J. Biomed. Sci.; 2003; 10; 337). Also known are methods of linking cationic polymers, such as, for example, PEI or dendrimers or cationic proteins, such as, for example, polylysine (Chen et al., FEBS Letters; 1994; 338; 167; Suh et al.; J. Controlled Release; 2001; 72; 171) covalently to antibodies or antibody fragments; PEI is reacted, for example, with DPS in DMSO and added to an antibody solution in PBS. After a dialysis, the non-viral gene delivery system coupled to antibody is available (Chiu et al., J. Controlled Release; 2004; 97; 357).

In a method according to the invention, the first solution can contain an agonist for activation of the TLR 7 and/or TLR 8, especially one of those mentioned above. The agonist can be dissolved in a buffered salt solution, for example PBS, basal medium, etc., having a physiological pH value of from 6.8 to 7.45 and a physiological osmolality of from 270 to 310 mosmol/kgH₂O, in water or in an unbuffered salt solution having a pH value of from 5 to 9. The concentration of the agonist can be from 0.01 to 1 μg/μl. That first solution containing a TLR 7 and/or TLR 8 agonist can be added to the cells to be transfected in the culture medium before, during or after the transfection. The time of agonist treatment should be selected in dependence upon the nature of the agonist so that an innate immune system that is as active as possible encounters as high as possible a number of siRNA. Preferably, the addition of the first solution containing the agonist is effected at the same time as the addition of the transfection complex to the cells to be transfected, by adding a corresponding amount of the first solution to the culture medium. The amount of agonist is governed by the amount of cells. Preferably, from 0.1 pg agonist/cell to 25 ng agonist/cell, especially from 1 to 500 pg agonist/cell, more especially from 10 to 250 pg agonist/cell, are used.

According to the invention, a method according to the invention, that is to say a transfection, can also be effected in vivo, the steps of the method corresponding to the respective steps of an in vitro transfection except that the addition of the respective solutions or mixtures takes place perorally (p.o.), percutaneously, sublingually (s.l.), nasally, intravenously (i.v.), intra-articularly, intra-arterially (i.a.), intralymphatically, intra-muscularly (i.m.), intra-ossally (i.o.), subcutaneously (s.c.), intracutaneously (i.c.), transdermally, rectally, vaginally, by inhalation (p.i.=per inhalation), intrapulmonally, endobronchially (e.b.), intraperitoneally (i.p.), intracardially, intraneurally, perineurally, peridurally, intrathecally, intrapleurally, intravitreally, parenterally, enterally or buccally. The amount of genetic material is governed by the nature of the genetic material, the target compartment, for example blood, extracellular fluid of the tissue, cell tissue, etc., the form of administration and the nature of the addition, for example infusion, injection or inhalation, and is from 0.01 to 500 mg/kg body weight. The amount of antibody, antagonist or agonist in the case of direct administration of a first solution to an individual can be from 0.1 mg/kg to 500 mg/kg body weight, preferably from 1 to 100 mg/kg, especially from 5 to 10 mg/kg. The amount of inhibitor in the case of direct administration of a first solution to an individual can be from 0.1 mg/kg to 500 mg/kg body weight, preferably from 10 to 300 mg/kg, especially from 100 to 200 mg/kg. The time and duration of the addition of a first solution is governed by the target compartment, for example blood, extracellular fluid of the tissue, cell tissue, etc., the form of administration and the mode of administration, for example infusion, injection, inhalation.

In the case of in vivo applications, the time interval between two transfections can be up to 6 weeks.

According to the invention there is provided a composition which comprises at least two of the following components:
a) a non-viral gene delivery system,
c) genetic material, and
b) a means for at least partially suppressing or activating the innate intracellular and/or intercellular immunity.

According to the invention there is also provided a kit of parts which comprises at least two of the following components:
a) a non-viral gene delivery system,
c) genetic material, and
b) a means for at least partially suppressing or activating the innate intracellular and/or intercellular immunity.

According to the invention, the non-viral gene delivery system comprises especially a cationic lipid, a cationic polymer, a cationic protein; and/or a compound which has a DNA- and/or RNA-binding domain and is able to trigger receptor-mediated endocytosis or a membrane transfer; and/or a compound which is covalently bound to DNA and/or RNA and is able to trigger receptor-mediated endocytosis or a membrane transfer.

According to the invention, the genetic material used can be, for example, genetic material for repairing a gene defect, for example genetic material, especially modified or unmodified ssDNA, modified or unmodified dsDNA, modified or unmodified ssRNA, modified or unmodified dsRNA and/or modified or unmodified siRNA.

According to the invention, the means used for at least partially suppressing and/or activating the innate intracellular and/or intercellular immunity can be at least one activity-reducing or activity-increasing effector.

Furthermore, according to the invention the means used for at least partially suppressing the innate intracellular and/or intercellular immunity can be an antibody, intrabody, aptamer, antagonist, inhibitor and/or an siRNA.

Preferably, the antibody, intrabody, aptamer, antagonist, inhibitor and/or siRNA used as means for at least partially suppressing the innate intracellular and/or intercellular immunity is able to block at least one signal transduction cascade of the innate immune system.

Furthermore, according to the invention the means used for at least partially activating the innate intracellular and/or intercellular immunity can be an agonist.

Preferably, the agonist used as means for at least partially activating the innate intracellular and/or intercellular immunity is able to activate at least one signal transduction cascade of the innate immune system.

The means according to the invention for at least partially suppressing the innate intracellular and/or intercellular immunity can also comprise genetic material which is able to effect a knock-down of a protein of a signal transduction cascade of the innate immune system.

Moreover, the means according to the invention for at least partially suppressing the innate intracellular and/or intercellular immunity can comprise genetic material which can lead to expression of a protein that is able to block the activity of a protein in a signal transduction cascade of the innate immune system.

Preferably, a composition according to the invention for a transfection can comprise (a) a non-viral gene delivery system and (b) a means for at least partially suppressing and/or activating the innate intracellular and/or intercellular immunity, it being possible for the non-viral gene delivery system:
(i) to comprise a cationic lipid, a cationic polymer or a cationic protein; and/or
(ii) to comprise a compound which has a DNA- and/or RNA-binding domain and is able to trigger receptor-mediated endocytosis or a membrane transfer; and/or
(iii) to comprise a compound which is covalently bound to DNA and/or RNA and is able to trigger receptor-mediated endocytosis or a membrane transfer;

and it being possible for the means for at least partially suppressing and/or activating the innate intracellular and/or intercellular immunity to be selected from:
(i) an antibody to TLR 1, TLR 2, TLR 3, TLR 4, TLR 5, TLR 6, TLR 7, TLR 8, TLR 9, TLR 10, TLR 11, TLR 12 or TLR 13;
(ii) an antibody to a cytokine receptor or a cytokine receptor antagonist;
(iii) an inhibitor of kinase MEK1 and/or MEK2;
(iv) an agonist for TLR7 and/or TLR8, selected from the group comprising bropirimine (2-amino-5-bromo-6-phenyl-4-pyrimidinone), imidazoquinolines, thiazoloquinolines and guanosine analogues; and
(v) a combination thereof.

A kit of parts according to the invention for transfection can comprise preferably (a) a non-viral gene delivery system and (b) a means for at least partially suppressing and/or activating the innate intracellular and/or intercellular immunity, it being possible for the non-viral gene delivery system:
(i) to comprise a cationic lipid, a cationic polymer or a cationic protein; and/or
(ii) to comprise a compound which has a DNA- and/or RNA-binding domain and is able to trigger receptor-mediated endocytosis or a membrane transfer; and/or
(iii) to comprise a compound which is covalently bound to DNA and/or RNA and is able to trigger receptor-mediated endocytosis or a membrane transfer;

and it being possible for the means for at least partially suppressing and/or activating the innate intracellular and/or intercellular immunity to be selected from:
(i) an antibody to TLR 1, TLR 2, TLR 3, TLR 4, TLR 5, TLR 6, TLR 7, TLR 8, TLR 9, TLR 10, TLR 11, TLR 12 or TLR 13;
(ii) an antibody to a cytokine receptor or a cytokine receptor antagonist;
(iii) an inhibitor of kinase MEK1 and/or MEK2;
(iv) an agonist for TLR7 and/or TLR8, selected from the group comprising bropirimine (2-amino-5-bromo-6-phenyl-4-pyrimidinone), imidazoquinolines, thiazoloquinolines and guanosine analogues; and
(v) a combination thereof.

More preferably, a composition according to the invention or a kit of parts according to the invention comprises a non-viral gene delivery system containing a cationic lipid.

Furthermore, a composition according to the invention or a kit of parts according to the invention can comprise a non-viral gene delivery system containing a cationic lipid in accordance with the following formula:

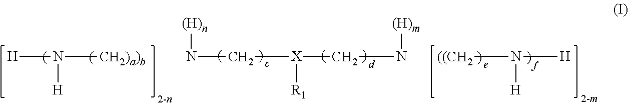

wherein

R₁ may be

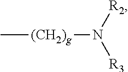

wherein

R₂ and R₃ are each independently of the other dodecyl, dodecenyl, tetradecyl, tetradecenyl, hexadecyl, hexadecenyl, octadecyl, octadecenyl or other alkyl radicals which, in all possible combinations, may be saturated, unsaturated, branched, unbranched, fluorinated or non-fluorinated and may be composed of from 5 to 30 carbon atoms;

X may be

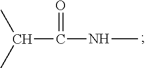

and wherein

m=0 and n=0; or m=0 and n=1; or m=0 and n=2; or m=1 and n=1; or m=1 and n=2; or m=2 and n=2; and g may be 1, 2, 3, 4, 5, 6, 7 or 8; a may be 0, 1, 2, 3, 4, 5 or 6; b may be 0, 1, 2, 3, 4, 5 or 6; c may be 0, 1, 2, 3, 4, 5 or 6; d may be 0, 1, 2, 3, 4, 5 or 6; e may be 0, 1, 2, 3, 4, 5 or 6, and f may be 0, 1,2, 3, 4, 5 or 6.

Preferably, in the cationic lipid R₂ and R₃ each independently of the other may be dodecyl, dodecenyl, tetradecyl, tetradecenyl, hexadecyl, hexadecenyl, octadecyl, octadecenyl; m and n may be 1; and g may be 1, 2, 3, 4, 5, 6, 7 or 8; a may be 0, 1, 2, 3, 4, 5 or 6; b may be 0, 1, 2, 3, 4, 5 or 6; c may be 0, 1, 2, 3, 4, 5 or 6; d may be 0, 1, 2, 3, 4, 5 or 6; e may be 0, 1, 2, 3, 4, 5 or 6, and f may be 0, 1,2, 3, 4, 5 or 6.

According to the invention, the composition or the kit of parts can contain modified or unmodified genetic material, especially modified or unmodified ssDNA, modified or unmodified dsDNA, modified or unmodified ssRNA, modified or unmodified dsRNA and/or modified or unmodified siRNA.

Preferably, the composition or the kit of parts can contain as means for at least partially suppressing and/or activating the innate intracellular and/or intercellular immunity 1,4-diamino-2,3-dicyano-1,4-bis(o-aminophenylmercapto)butadiene (U0126); imiquimod (R837, 1-(2-methylpropyl)-1H-imidazo[4,5-c]quinoline-4-amine); resiquimod (R848, 4-amino-2-(ethoxymethyl)-a,a-dimethyl-1H-imidazo[4,5-c]quinoline-1-ethanol); gardiquimod (1-(4-amino-2-ethylaminomethylimidazo[4,5-c]quinolin-1-yl)-2-methylpropan-2-ol); CL075; CL097; loxoribine (7-allyl-7,8-dihydro-8-oxo-guanosine); isatoribine (7-thia-8-oxo-guanosine); bropirimine (2-amino-5-bromo-6-phenyl-4-pyrimidinone); or any combination thereof.

Furthermore, the composition or the kit of parts can contain as means for at least partially suppressing and/or activating the innate intracellular and/or intercellular immunity an antibody to TLR 3, TLR 7, TLR 8 or TLR 9.

Moreover, according to the invention the composition or the kit of parts can contain as means for at least partially suppressing and/or activating the innate intracellular and/or intercellular immunity an antibody or antagonist against interleukin-1-receptors, especially IL-ra; interferon-typed-receptors; interferon-gamma-receptors; or tumour necrosis factor receptors.

In a preferred embodiment, a plurality of the above-mentioned means for at least partially suppressing and/or activating the immunity can be combined with one another, that is to say two, three or more components can be used in the means according to the invention for at least partially suppressing and/or activating the innate intracellular and/or intercellular immunity. It is likewise possible for a plurality of the above-mentioned components a) and/or b) and/or c) to be used.

In the kit of parts according to the invention it is possible for:
all components to be present separately from one another, or
components a) and b) to be present together, or
components a) and c) to be present together, or
components b) and c) to be present together.

Preferably, in a kit of parts it is possible for:
all components to be present entirely separately from one another, or
components a) and b) to be present separately from one another, or
components a) and b) to be present together.

For example, the components can be present either separately from one another, for example in glass or plastics containers which are packaged together, or two components together or several components together can be provided in suitable containers.

According to the invention, the kit of parts can contain the non-viral gene delivery system in the form of salts, especially formulated as liposomes/micelles; in the form of a solution, especially in the form of an aqueous solution, salt solution, buffered salt solution, for example salt solution with HEPES buffer, or solution in a physiologically acceptable solvent, for example ethanol/DMSO; in lyophilised form; in the form of a solid or in the form of a film. Solutions of the non-viral gene delivery system preferably have a pH value of from 5 to 9. The final concentration of the non-viral gene delivery system in the culture medium is preferably from 0.01 to 10 mg/ml.

The means for at least partially suppressing and/or activating the innate intracellular and/or intercellular immunity, such as, for example, an antibody, antagonist or inhibitor, can be contained in a kit of parts according to the invention in lyophilised form; dissolved in water, salt solution, buffered salt solution, for example salt solution with PBS buffer, or a suitable organic solvent, for example ethanol, DMSO, etc. Solutions preferably have a pH value of from 5 to 9. Optionally stabilisers may be present. The means for at least partially suppressing and/or activating the innate intracellular and/or intercellular immunity is preferably present in a concentration of from 0.01 to 10 mg/ml.

Kits containing genetic material can contain that genetic material in lyophilised form, dissolved in water, salt solution or buffered salt solution, for example salt solution with TE buffer. Solutions of the genetic material preferably have a pH value of from 5 to 9. The final concentration of the genetic material in the culture medium is preferably from 0.01 to 10 mg/ml.

The composition according to the invention and/or the kit of parts according to the invention can be used for carrying out a method according to the invention.

Furthermore, the composition according to the invention can be in the form of a pharmaceutical composition and the kit of parts according to the invention can be in the form of a pharmaceutical kit of parts.

Moreover, a composition or kit of parts according to the invention can be used in the treatment of a disease by gene therapy.

The disease may be, for example, cystic fibrosis, muscular dystrophy, phenylketonuria, maple syrup disease, propionazidaemia, methylmalonazidaemia, adenosine deaminase deficiency, hypercholesterolaemia, haemophilia, β-thalassaemia, cancer, a viral disease, macular degeneration, amyotrophic lateral sclerosis and/or an inflammatory disease.

The present invention can also be used for carrying out transfections without the expression profile of the cells being changed to an undesirable extent. That is of special interest in in vivo applications, where the activation of the immune system frequently presents a problem.

Unnecessary changes to the remaining expression profile of the cell are particularly undesirable also in the case of the investigation of signal transduction pathways by the knock-down of a participating protein by siRNA, especially since it is known that the signal transduction cascades frequently affect one another.

### Examples

General Material:

1. Hela cells

2. Metafectene Pro, T040-1.0, Biontex Laboratories

3. 24-well plate, TPP, Product No. 92024

4. 48-well plates, Corning Inc., Costar, Product No. 3548

5. DMEM, PAA, Cat. No. E15-883

6. FCS Mycoplex, PAA, Cat. No. E15-773

7. pCMV-lacZ, Product No. PF462-060207, PlasmidFactory, c=1 mg/ml in WFI

8. β-Galactosidase Assay Kit, Stratagene

9. Hela-Luc cells (cells stably transfected with luciferase)

10. Luciferase Assay Kit, Promega

11. BCA Protein Assay Kit, Thermo Scientific, Product No. 23227

12. dimethyl sulfoxide (DMSO for molecular biology), Fluka; No. 41639

13. siRNA, desalted, 30 pMol/μl in Universal Buffer (siMAX from MWG) directed against luciferase GL3 (anti-luciferase-siRNA):

| | |
|---|---|
| Sense Sequence: | CUUACGCUGAGUACUUCGAtt |
| Antisense Sequence: | UCGAAGUACUCAGCGUAAGtt |
| Non-specific control: | |
| Sense Sequence: | AGGUAGUGUAAUCGCCUUGtt |
| Antisense Sequence: | CAAGGCGAUUACACUACCUtt |

### Example 1

Material:

1. Sheep Polyclonal Antibody against human IFNβ, PBL Biomedical Laboratories, Product Number 31400-1, 0.25 mg/ml (estimate), 2×10⁴ units/ml.

Detailed Description of Experiment:

1st day: HeLa cells are sown in a 24-well plate, the cells being plated out at a cell count of 2.3*10⁵ cells per well of 500 μl of complete medium (10% FCS). Incubation is then carried out in a CO₂ incubator (10%) for 24 hours.

2nd Day:

First of all the antibody (Sheep Polyclonal Antibody against human IFNI3) is thawed. 400 μl of PBS (phosphate buffered saline) are then added thereto and gentle mixing is carried out. The antibody now has a concentration of 0.05 μg/μl. The wells of the 24-well plate are then supplied with the following amounts of antibody:

| | 1 | 2 | 3 | 4 | 5 | 6 |
|---|---|---|---|---|---|---|
| A | 0 μg (0 μl) | 0.25 μg (5 μl) | 0.5 μg (10 μl) | 1 μg (20 μl) | 1.5 μg (30 μl) | 3 μg (60 μl) |
| B | 0 μg (0 μl) | 0.25 μg (5 μl) | 0.5 μg (10 μl) | 1 μg (20 μl) | 1.5 μg (30 μl) | 3 μg (60 μl) |
| C | 0 μg (0 μl) | 0.25 μg (5 μl) | 0.5 μg (10 μl) | 1 μg (20 μl) | 1.5 μg (30 μl) | 3 μg (60 μl) |
| D | 0 μg (0 μl) | 0.25 μg (5 μl) | 0.5 μg (10 μl) | 1 μg (20 μl) | 1.5 μg (30 μl) | 3 μg (60 μl) |

Incubation is then carried out in an incubator for 0.5 hour. In the meantime, the lipoplexes are prepared. For that purpose, 26 μl of DNA (pCMV-lacZ) are pipetted into 1300 μl of PBS and mixed by gently pipetting up and down. 104 μl of Metafectene Pro are likewise pipetted into 1300 μl of PBS and mixed by gently pipetting up and down. The two solutions are then mixed and incubated for 15 min.

Finally, 100 μl of the lipoplex solution are added to each well. Incubation is then carried out in a CO₂ incubator (10%) for 24 hours.

3rd Day:

On the third day the medium of lines C and D is renewed. The steps of lipoplex preparation and transfection are repeated for those lines. Incubation is then carried out in a CO₂ incubator (10%) for 24 hours.

4th Day

Reporter Gene Assay:

The efficiency of the transfection is obtained using the β-Galactosidase Assay Kit in accordance with the manufacturer's instructions. The plates are developed until a yellow colouring with an absorption of 1-2 is measured in the microplate reader and then immediately stopped. The incubation time is then noted. The values are read-out using the microplate reader and the average value is formed.

Result:

Incubation time 9.5 min

Average Value of 3 Measurements:

| | 1 | 2 | 3 | 4 | 5 | 6 |
|---|---|---|---|---|---|---|
| A | 0.717 | 0.689 | 0.650 | 0.652 | 0.635 | 0.509 |
| B | 0.668 | 0.672 | 0.690 | 0.679 | 0.714 | 0.525 |
| C | 0.858 | 0.823 | 1.228 | 1.690 | 1.852 | 0.975 |
| D | 1.501 | 1.805 | 1.518 | 1.886 | 2.017 | 1.909 |

Lines A and B are duplicates of the results for a single transfection. Lines C and D are duplicates of the results for a repetitive transfection. The average value is therefore formed:

| | 1 | 2 | 3 | 4 | 5 | 6 |
|---|---|---|---|---|---|---|
| A&B/2 | 0.6925 | 0.6805 | 0.6700 | 0.6655 | 0.6745 | 0.5170 |
| C&D/2 | 1.1810 | 1.3140 | 1.3730 | 1.7880 | 1.9345 | 1.4420 |

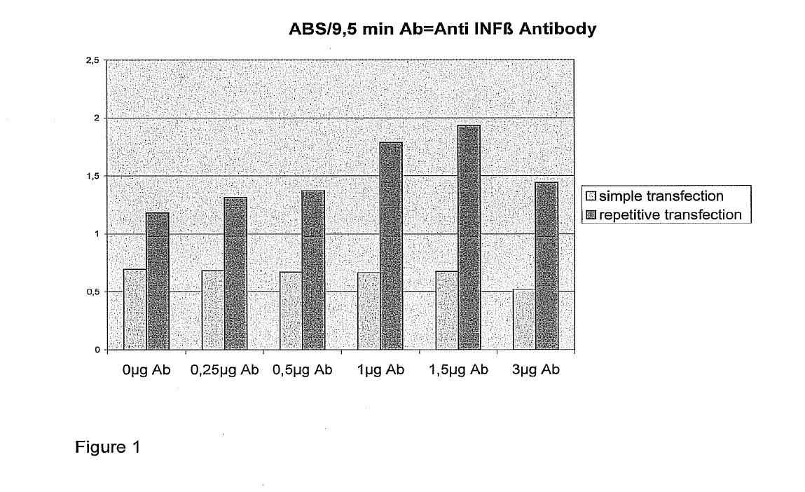

See FIG. 1

### Example 2

Material: 1. Mouse monoclonal Antibody against Human Interferon Alpha/Beta Receptor Chain 2 (CD118), clone MMHAR-2, isotype Ig2a, C=0.5 mg/ml in PBS (phosphate buffered saline) containing 0.1% bovine serum albumin (BSA), PBL Biomedical Laboratories, Product No. 21385.

Detailed Description of Experiment:

### Analogous to Example 1

Differences:

First of all 400 μl of PBS (phosphate buffered saline) are added to the antibody (Mouse monoclonal Antibody against Human Interferon Alpha/Beta Receptor Chain) and gentle mixing is carried out. The antibody now has a concentration of 0.1 μg/μl. The wells of the 24-well plate are supplied with the following amounts of antibody:

| | 1 | 2 | 3 | 4 | 5 | 6 |
|---|---|---|---|---|---|---|
| A | 0 μg (0 μl) | 0.5 μg (5 μl) | 1 μg (10 μl) | 2 μg (20 μl) | 3 μg (30 μl) | 6 μg (60 μl) |
| B | 0 μg (0 μl) | 0.5 μg (5 μl) | 1 μg (10 μl) | 2 μg (20 μl) | 3 μg (30 μl) | 6 μg (60 μl) |
| C | 0 μg (0 μl) | 0.5 μg (5 μl) | 1 μg (10 μl) | 2 μg (20 μl) | 3 μg (30 μl) | 6 μg (60 μl) |
| D | 0 μg (0 μl) | 0.5 μg (5 μl) | 1 μg (10 μl) | 2 μg (20 μl) | 3 μg (30 μl) | 6 μg (60 μl) |

The addition of lipoplex takes place after 5 hours' incubation time with the antibody.

Result:

Incubation time: 4 min

Average Value of 3 Measurements:

| | 1 | 2 | 3 | 4 | 5 | 6 |
|---|---|---|---|---|---|---|
| A | 1.269 | 1.113 | 1.163 | 1.185 | 1.214 | 1.084 |
| B | 1.132 | 1.102 | 1.088 | 1.229 | 1.113 | 1.154 |
| C | 1.127 | 1.438 | 1.533 | 1.378 | 1.504 | 1.367 |
| D | 1.395 | 1.393 | 1.377 | 1.417 | 1.459 | 1.467 |

Lines A and B are duplicates of the results for a single transfection. Lines C and D are duplicates of the results for a repetitive transfection. The average value is therefore formed:

| | 1 | 2 | 3 | 4 | 5 | 6 |
|---|---|---|---|---|---|---|
| A&B/2 | 1.2005 | 1.1075 | 1.1255 | 1.2070 | 1.1635 | 1.1190 |
| C&D/2 | 1.2610 | 1.4155 | 1.4550 | 1.3975 | 1.4815 | 1.4170 |

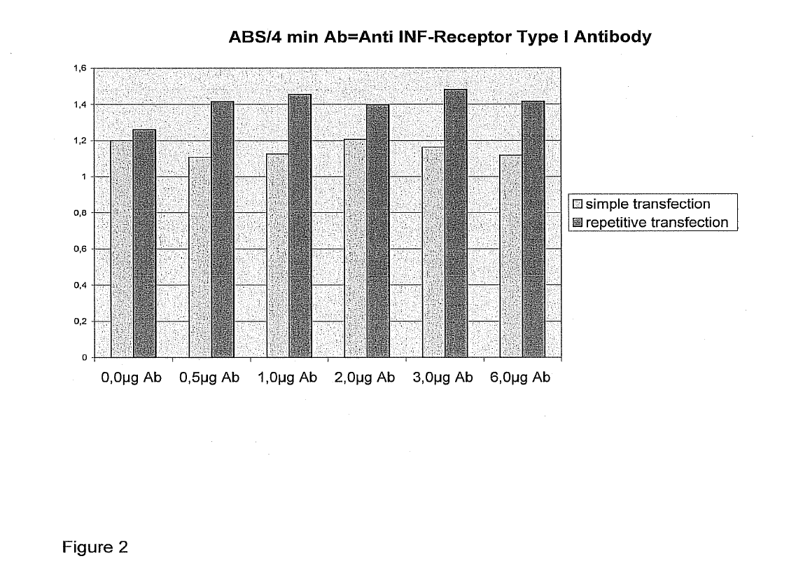

See FIG. 2

### Example 3

Further experiments with antibodies to receptors and cytokines.

Detailed Description of Experiment:

1st day HeLa cells are sown in a 48-well plate, the cells being plated out at a cell count of 1.2*10⁵ cells per well of 250 μl of complete medium (10% FCS). Incubation is then carried out in a CO₂ incubator (10%) for 24 hours.

2nd Day:

The antibody is adjusted to a concentration of 0.05 μg/μl with PBS. The wells of the 48-well plate are then supplied with the following amounts of antibody:

| | 1 | | 2 | | 3 | | 4 | |
|---|---|---|---|---|---|---|---|---|
| A | 0 μg | (0 μl) | 0 μg | (0 μl) | 0 μg | (0 μl) | 0 μg | (0 μl) |
| B | 0.25 μg | (5 μl) | 0.25 μg | (5 μl) | 0.25 μg | (5 μl) | 0.25 μg | (5 μl) |
| C | 0.5 μg | (10 μl) | 0.5 μg | (10 μl) | 0.5 μg | (10 μl) | 0.5 μg | (10 μl) |
| D | 1 μg | (20 μl) | 1 μg | (20 μl) | 1 μg | (20 μl) | 1 μg | (20 μl) |
| E | 1.5 μg | (30 μl) | 1.5 μg | (30 μl) | 1.5 μg | (30 μl) | 1.5 μg | (30 μl) |
| F | 3 μg | (60 μl) | 3 μg | (60 μl) | 3 μg | (60 μl) | 3 μg | (60 μl) |

Incubation is then carried out in an incubator for 5 hours or 0.5 hour, depending upon the antibody. The lipoplexes are then prepared. For that purpose, 13 μl of DNA (pCMV-lacZ) are pipetted into 650 μl of PBS and mixed by gently pipetting up and down. 52 μl of Metafectene Pro are likewise pipetted into 650 μl of PBS and mixed by gently pipetting up and down. The two solutions are then mixed and incubated for 15 min. Finally, 50 μl of the lipoplex solution are added to each well. Incubation is then carried out in a CO₂ incubator (10%) for 24 hours.

3rd Day:

On the third day the medium of columns 3 and 4 is renewed. The steps of lipoplex preparation and transfection are repeated for those lines. Incubation is then carried out in a CO₂ incubator (10%) for 24 hours.

4th Day:

Reporter Gene Assay:

Analogous to Example 1 (with half amounts)

Results:

Antibody:

Mouse Anti-human-CD282 Antibody (=anti-TLR2), monoclonal, AbD Serotec, Cat. No.: MC2484EL

Pre-incubation time with the antibody prior to transfection: 5 hours

Development time reporter gene assay [min]: 5

Average Value of 3 Measurements:

| | 1 | 2 | 3 | 4 |
|---|---|---|---|---|
| A | 0.65 | 0.66 | 1.08 | 1.18 |
| B | 0.61 | 0.68 | 1.12 | 1.07 |
| C | 0.72 | 0.70 | 1.70 | 1.21 |
| D | 0.67 | 0.76 | 1.82 | 1.34 |
| E | 0.67 | 0.86 | 1.62 | 1.66 |
| F | 0.64 | 0.67 | 1.56 | 1.34 |

Columns 1 and 2 are duplicates of the results for a single transfection. Columns 3 and 4 are duplicates of the results for a repetitive transfection. The average value is therefore formed:

| | A | B | C | D | E | F |
|---|---|---|---|---|---|---|
| 1&2/2 | 0.65 | 0.64 | 0.71 | 0.71 | 0.76 | 0.65 |
| 3&4/2 | 1.13 | 1.09 | 1.45 | 1.58 | 1.65 | 1.45 |

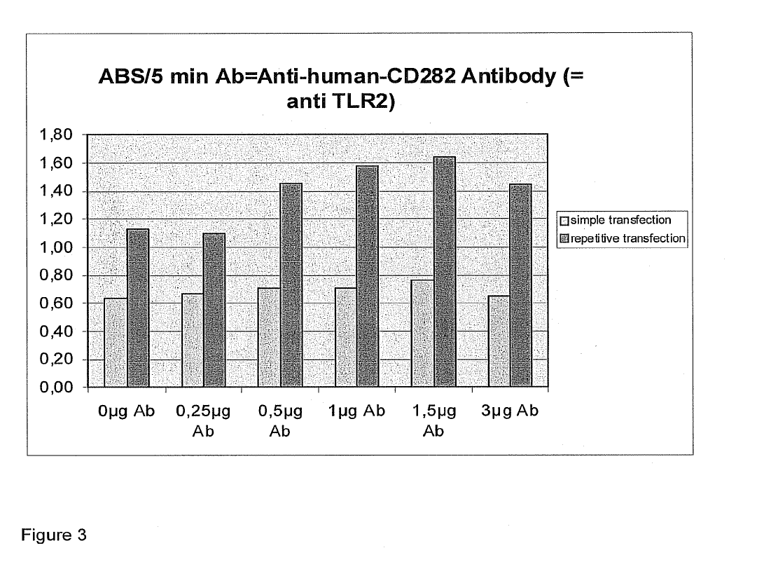

See FIG. 3

Antibody:

Mouse Anti-human TLR3 Antibody, monoclonal, Lifespan Biosciences Cat. No. LS-C18685

Pre-incubation time with the antibody prior to transfection: 5 hours

Development time reporter gene assay [min]: 5.5

Average Value of 3 Measurements:

| | 1 | 2 | 3 | 4 |
|---|---|---|---|---|
| A | 0.72 | 0.64 | 1.30 | 1.32 |
| B | 0.64 | 0.67 | 1.42 | 1.30 |
| C | 0.75 | 0.73 | 1.54 | 1.59 |
| D | 0.75 | 0.86 | 1.71 | 1.69 |
| E | 0.89 | 0.82 | 1.65 | 1.95 |
| F | 0.76 | 0.82 | 1.83 | 1.65 |

Columns 1 and 2 are duplicates of the results for a single transfection. Columns 3 and 4 are duplicates of the results for a repetitive transfection. The average value is therefore formed:

| | A | B | C | D | E | F |
|---|---|---|---|---|---|---|
| 1&2/2 | 0.68 | 0.65 | 0.74 | 0.80 | 0.85 | 0.79 |
| 3&4/2 | 1.31 | 1.36 | 1.56 | 1.70 | 1.80 | 1.74 |

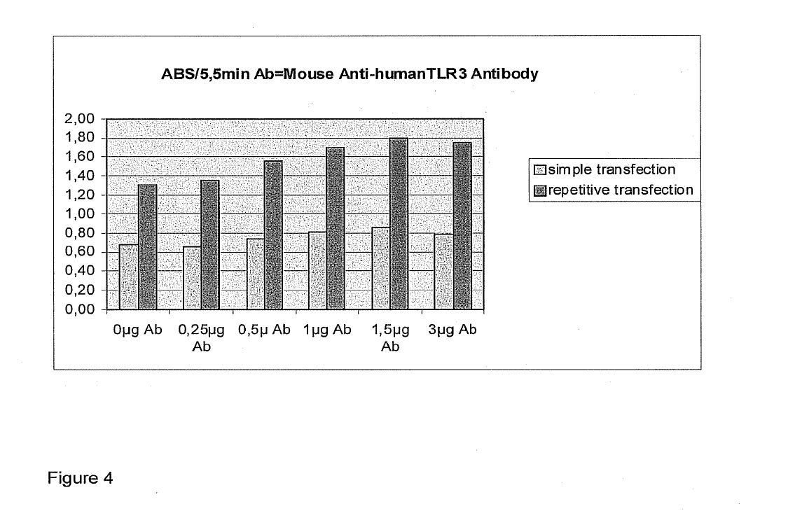

See FIG. 4

Antibody:

Mouse Anti-human-CD284 Antibody (=anti TLR4), monoclonal, AbD Serotec. Cat. No. MCA2061EL

Pre-incubation time with the antibody prior to transfection: 5 hours

Development time reporter gene assay [min]: 5.5

Average Value of 3 Measurements:

| | 1 | 2 | 3 | 4 |
|---|---|---|---|---|
| A | 0.65 | 0.65 | 1.03 | 1.22 |
| B | 0.67 | 0.65 | 1.44 | 1.67 |
| C | 0.74 | 0.64 | 1.42 | 1.17 |
| D | 0.78 | 0.69 | 1.57 | 1.38 |
| E | 0.74 | 0.73 | 1.59 | 1.63 |
| F | 0.78 | 0.71 | 1.82 | 1.56 |

Columns 1 and 2 are duplicates of the results for a single transfection. Columns 3 and 4 are duplicates of the results for a repetitive transfection. The average value is therefore formed:

| | A | B | C | D | E | F |
|---|---|---|---|---|---|---|
| 1&2/2 | 0.65 | 0.66 | 0.69 | 0.73 | 0.73 | 0.74 |
| 3&4/2 | 1.12 | 1.55 | 1.29 | 1.47 | 1.61 | 1.69 |

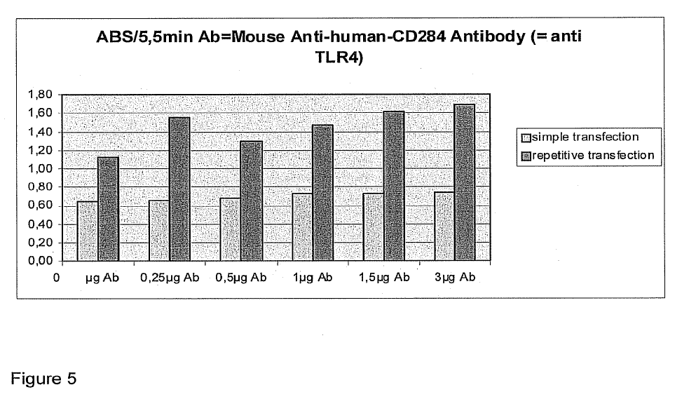

See FIG. 5

Antibody:

Antibody: Mouse Anti-human TLR1 Antibody (monoclonal, 0.05% sodium azide, 100 μg lyophilisate); Invivogen, No. Mab-htlr1.

For removal of the sodium azide the antibody was dialysed against PBS:

Dialysis membrane: Spectra/Por DispoDialyser (500 μl, cellulose ester membrane, 25000 Da molecular weight cut-off); Spectrum Laboratories; No. 135492, Lot 3224004.

Pre-incubation time with the antibody prior to transfection: 5 hours

Development time reporter gene assay [min]: 4

Average Value of 3 Measurements:

| | 1 | 2 | 3 | 4 |
|---|---|---|---|---|
| A | 1.055 | 1.225 | 1.683 | 1.627 |
| B | 1.354 | 1.328 | 1.905 | 1.798 |
| C | 1.346 | 1.516 | 2.033 | 1.914 |
| D | 1.401 | 1.367 | 2.106 | 1.827 |
| E | 1.260 | 1.488 | 2.063 | 1.841 |
| F | 1.085 | 1.020 | 1.816 | 1.712 |

Columns 1 and 2 are duplicates of the results for a single transfection. Columns 3 and 4 are duplicates of the results for a repetitive transfection. The average value is therefore formed:

| | A | B | C | D | E | F |
|---|---|---|---|---|---|---|
| 1&2/2 | 1.140 | 1.341 | 1.431 | 1.384 | 1.374 | 1.053 |
| 3&4/2 | 1.655 | 1.852 | 1.974 | 1.966 | 1.952 | 1.764 |

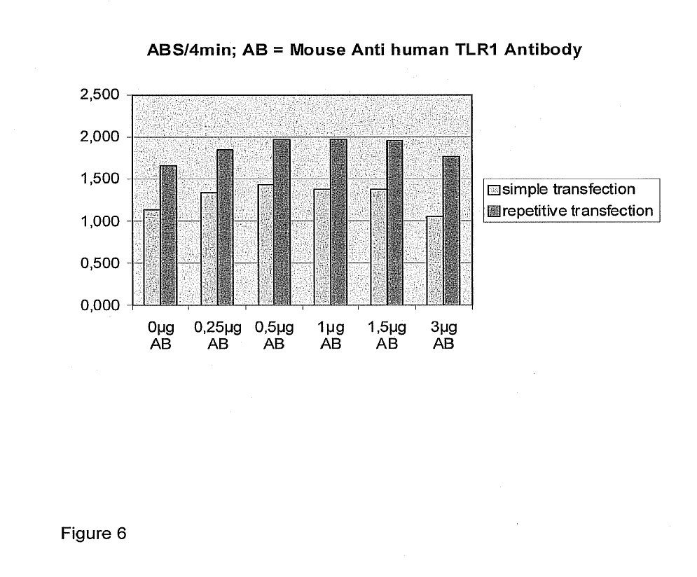

See FIG. 6

### Example 4

Further experiments with antagonists.

### Detailed Description of Experiment:

1st day: Analogous to Example 3.

2nd Day:

The antagonist is adjusted to a concentration of 0.025 μg/μl with PBS. The wells of the 48-well plate are then supplied with the following amounts of antibody:

| | 1 | | 2 | | 3 | | 4 | |
|---|---|---|---|---|---|---|---|---|
| A | 0 μg | (0 μl) | 0 μg | (0 μl) | 0 μg | (0 μl) | 0 μg | (0 μl) |
| B | 0.0625 μg | (2.5 μl) | 0.0625 μg | (2.5 μl) | 0.0625 μg | (2.5 μl) | 0.0625 μg | (2.5 μl) |
| C | 0.125 μg | (5 μl) | 0.125 μg | (5 μl) | 0.125 μg | (5 μl) | 0.125 μg | (5 μl) |
| D | 0.250 μg | (10 μl) | 0.250 μg | (10 μl) | 0.250 μg | (10 μl) | 0.250 μg | (10 μl) |
| E | 0.375 μg | (15 μl) | 0.375 μg | (15 μl) | 0.375 μg | (15 μl) | 0.375 μg | (15 μl) |
| F | 0.75 μg | (30 μl) | 0.75 μg | (30 μl) | 0.75 μg | (30 μl) | 0.75 μg | (30 μl) |

Incubation is then carried out in an incubator for 7 hours. The lipoplexes are then prepared. For that purpose, 13 μl of DNA (pCMV-lacZ) are pipetted into 650 μl of PBS and mixed by gently pipetting up and down. 52 μl of Metafectene Pro are likewise pipetted into 650 μl of PBS and mixed by gently pipetting up and down. The two solutions are then mixed and incubated for 15 min. Finally, 50 μl of the lipoplex solution are added to each well. Incubation is then carried out in a CO₂ incubator (10%) for 24 hours.

3rd Day:

Analogously to Example 3.

4th Day:

Reporter Gene Assay:

Analogous to Example 1 (with half amounts)

Results:

Antagonist:

Human Interleukin-1 Receptor Antagonist (IL1-ra Human), Biomol, Cat. No.: 54592

Pre-incubation time with the antibody prior to transfection: 7 hours

Development time reporter gene assay [min]: 10

Average Value of 3 Measurements:

| | 1 | 2 | 3 | 4 |
|---|---|---|---|---|
| A | 0.436 | 0.433 | 0.932 | 0.820 |
| B | 0.427 | 0.448 | 0.791 | 1.021 |
| C | 0.415 | 0.435 | 0.856 | 1.185 |
| D | 0.393 | 0.439 | 1.035 | 1.026 |
| E | 0.392 | 0.460 | 1.076 | 1.081 |
| F | 0.443 | 0.455 | 1.161 | 1.207 |

Columns 1 and 2 are duplicates of the results for a single transfection. Columns 3 and 4 are duplicates of the results for a repetitive transfection. The average value is therefore formed:

| | A | B | C | D | E | F |
|---|---|---|---|---|---|---|
| 1&2/2 | 0.435 | 0.437 | 0.425 | 0.416 | 0.426 | 0.449 |
| 3&4/2 | 0.876 | 0.906 | 1.020 | 1.030 | 1.078 | 1.184 |

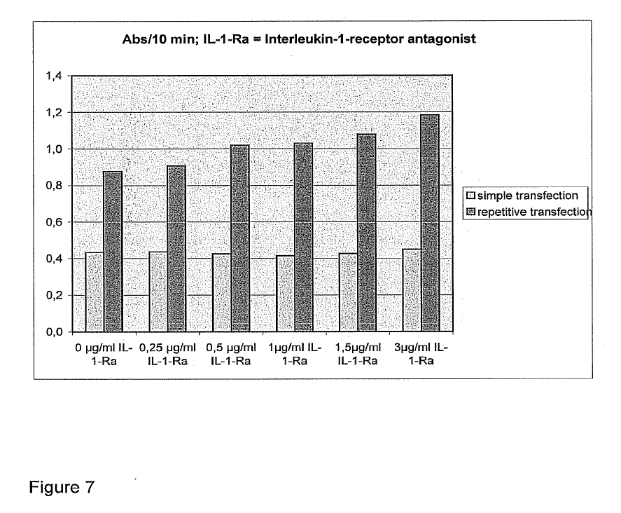

See FIG. 7

### Example 5

Further Experiment with a Kinase Inhibitor:

MEK1 and MEK2 Inhibitors:

U0126, 1,4-diamino-2,3-dicyano-1,4-bis(o-aminophenylmercapto)butadiene, MW=380.5, Invivogen, Cat. No.: tlr-u0126.

Detailed Description of Experiment:

1st day: HeLa cells are sown in a 48-well plate, the cells being plated out at a cell count of 0.8*10⁵ cells per well of 250 μl of complete medium (10% FCS). Incubation is then carried out in a CO₂ incubator (10%) for 24 hours.

2nd Day:

1 mg of inhibitor is dissolved in 100 μl of DMSO (stock solution). Dilution is then carried out with PBS 1:20 (working solution). The wells of the 48-well plate are then supplied with the following amounts of inhibitor:

| | F | E | D | C | B | A |
|---|---|---|---|---|---|---|
| 1 | 0 μm (0 μl) | 5 μM (0.95 μl) | 10 μM (1.9 μl) | 25 μM (4.75 μl) | 50 μM (9.5 μl) | 75 μM (14.25 μl) |

Incubation is then carried out in an incubator for 2 hours. The lipoplexes are then prepared. For that purpose, 5 μl of DNA (pCMV-lacZ) are pipetted into 250 μl of PBS and mixed by gently pipetting up and down. 20 μl of Metafectene Pro are likewise pipetted into 250 μl of PBS and mixed by gently pipetting up and down. The two solutions are then mixed and incubated for 15 min.

Finally, 50 μl of the lipoplex solution are added to each well. Incubation is then carried out in a CO₂ incubator (10%) for 48 hours.

3rd Day:

On the third day the medium is renewed and the amounts of inhibitor indicated in the table replaced. The steps of lipoplex preparation and transfection are repeated without an incubation period. Incubation is then carried out in a CO₂ incubator (10%) for 24 hours.

4th Day:

Reporter Gene Assay:

Analogous to Example 1 (with half amounts)

Results:

Pre-incubation time with the inhibitor prior to transfection: 2 hours

Development time reporter gene assay [min]: 10

Average Value of 3 Measurements:

| | F | E | D | C | B | A |
|---|---|---|---|---|---|---|
| 1 | 0.63 | 0.72 | 0.92 | 0.79 | 0.90 | 0.97 |

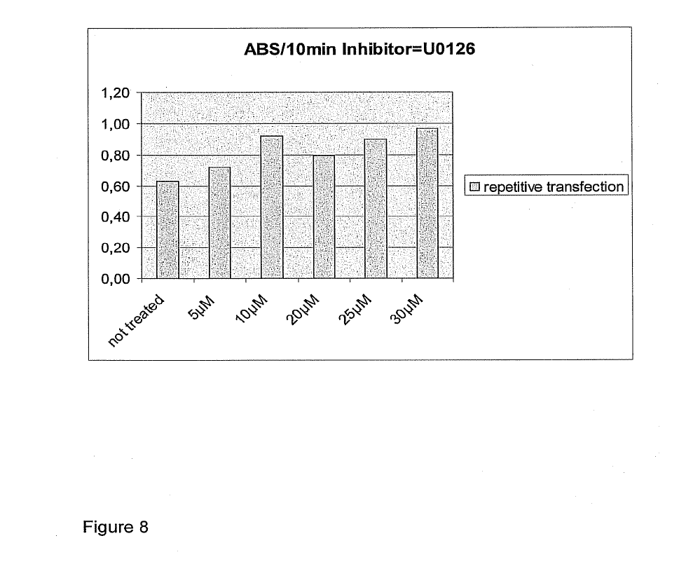

See FIG. 8

### Example 6

Further Experiment with HepG2 Cells

Procedure Analogous to Example 3

Differences: HepG2 cells, antibody concentration 0.1 μg/μl instead of 0.05 μg/μl in PBS, lipoplex formation in serum-free DMEM instead of in PBS.

Antibody:

Mouse Anti-human-CD282 Antibody (=anti TLR2), monoclonal, AbD Serotec, Cat. No.: MC2484EL

Pre-incubation time with the antibody prior to transfection: 5 hours

Development time reporter gene assay [min]: 4

Average Value of 3 Measurements:

| | 1 | 2 | 3 | 4 |
|---|---|---|---|---|
| A | 1.680 | 1.486 | 1.649 | 1.815 |
| B | 1.884 | 1.792 | 1.999 | 2.267 |
| C | 2.095 | 1.960 | 2.111 | 2.297 |
| D | 2.036 | 1.936 | 1.911 | 2.184 |
| E | 1.964 | 1.930 | 1.987 | 2.164 |
| F | 1.719 | 1.935 | 1.930 | 2.224 |

Columns 1 and 2 are duplicates of the results for a single transfection. Columns 3 and 4 are duplicates of the results for a repetitive transfection. The average value is therefore formed:

| | A | B | C | D | E | F |
|---|---|---|---|---|---|---|
| 1&2/2 | 1.583 | 1.838 | 2.028 | 1.986 | 1.947 | 1.827 |
| 3&4/2 | 1.732 | 2.133 | 2.204 | 2.048 | 2.076 | 2.077 |

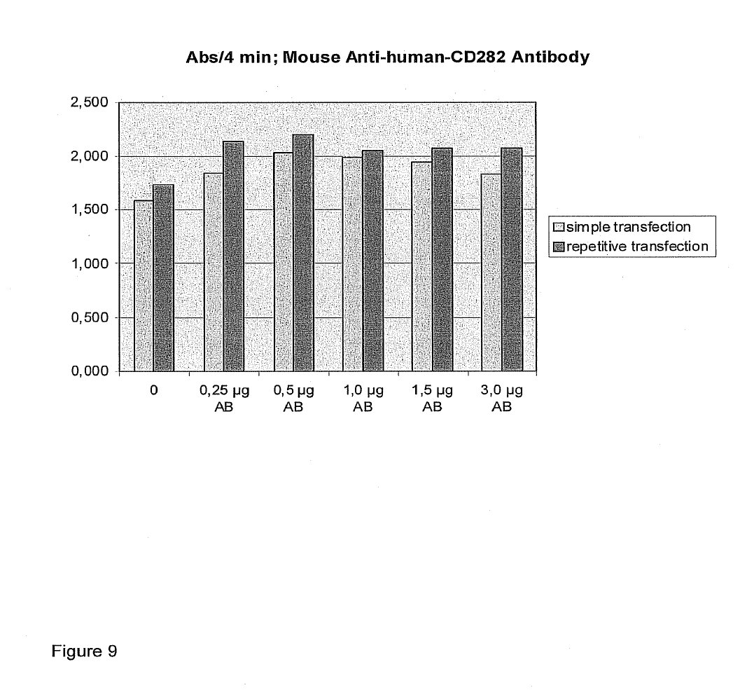

See FIG. 9

### Example 7

Further experiment with an antibody to TLR9 with incorporation into a lipoplex.

Antibody:

Rabbit Anti-human-TLR9 Antibody (=anti TLR9), polyclonal, 0.5 μg/μl in PBS, 0.2% gelatin, 0.05% sodium azide, Lifespan Biosciences Cat. No.: MCA2484EL.

For removal of the sodium azide the antibody was dialysed against PBS:

Dialysis membrane: Spectra/Por DispoDialyser (500 μl, cellulose ester membrane, 25000 Da molecular weight cut-off); Spectrum Laboratories; No. 135492, Lot 3224004.

Detailed Description of Experiment:

1st day: HeLa cells are sown in a 48-well plate, the cells being plated out at a cell count of 1.0*10⁵ cells per well of 250 μl of complete medium (10% FCS). Incubation is then carried out in a CO₂ incubator (10%) for 24 hours.

2nd Day:

The antibody is adjusted to a concentration of 0.1 μg/μl with PBS. 15 mg of DNA (pCMVβGal) and 60 μl of Metafectene Pro are each dissolved in 300 μl of serum-free DMEM. The individual solutions are mixed by gentle pipetting up and down. Lipoplexes of the following composition are then prepared.

| Solution | 1 | 2 | 3 | 4 | 6 |
|---|---|---|---|---|---|
| Antibody solution [μl] | 0 | 11 | 22 | 44 | 66 |
| Metafectene Pro solution [μl] | 44 | 44 | 44 | 44 | 44 |
| DNA solution [μl] | 44 | 44 | 44 | 44 | 44 |
| Serum-free DMEM [μl] | 132 | 121 | 110 | 88 | 66 |

The Metafectene Pro solution is first combined with the antibody solution and incubated for five minutes at room temperature (RT). The DNA solution and the serum-free DMEM is then added and incubation is carried out for a further 15 minutes at RT.

50 μl of the DNA-antibody-lipid complexes are pipetted into each of the wells, with solution 1 being pipetted into four wells of column 1, solution 2 into four wells of column 2 and so on. Incubation is then carried out in a CO₂ incubator (10%) for 24 hours.

3rd Day:

The medium of all lines is renewed. For lines C and D a second transfection is carried out analogously to the transfection on the first day.

4th Day:

Reporter Gene Assay:

Analogous to Example 1 (with half amounts)

Results:

Average Value of 3 Measurements:

| | 1 | 2 | 3 | 4 |
|---|---|---|---|---|
| A | 0.819 | 1.671 | 1.419 | 1.845 |
| B | 0.860 | 1.591 | 1.604 | 1.858 |
| C | 1.005 | 2.185 | 1.940 | 2.267 |
| D | 1.472 | 2.650 | 2.264 | 2.401 |

Columns 1 and 2 are duplicates of the results for a single transfection. Columns 3 and 4 are duplicates of the results for a repetitive transfection. The average value is therefore formed:

| | 1 | 2 | 3 | 4 |
|---|---|---|---|---|
| A&B | 0.840 | 1.631 | 1.512 | 1.852 |
| C&D | 1.239 | 2.417 | 2.102 | 2.334 |

The added amount of antibody corresponds to the following amounts of antibody per well:

| | 1 | 2 | 3 | 4 |
|---|---|---|---|---|
| μg | 0 | 0.5 | 1.0 | 1.5 |

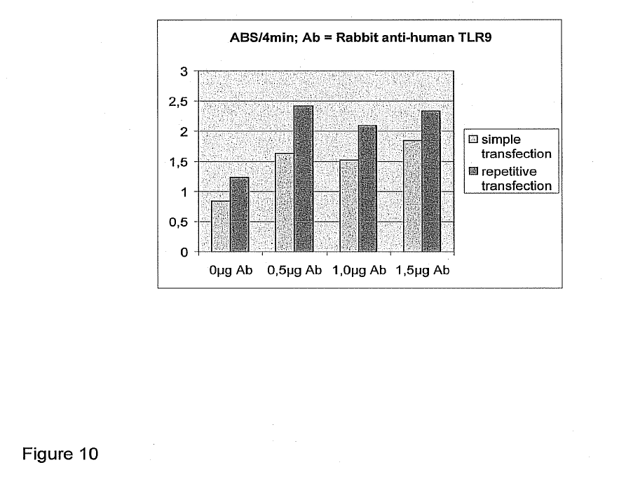

See FIG. 10

### Example 8

siRNA transfection in the presence of kinase inhibitors:
p38/RK MAPK inhibitor: SB203580, Invivogen; No. tirl-sb20
MEK inhibitor: U0126, Invivogen, No. tlr-u0126

Detailed Description of Experiment:

1st day: HeLa-Luc cells are sown in a 48-well plate, the cells being plated out at a cell count of 1.5×10⁴ cells per well of 250 μl of complete medium (10% FCS). Incubation is then carried out in a CO₂ incubator (10%) for 24 hours.

2nd Day:

First the inhibitors are prepared:

SB203580 (M=377.43 g/mol): 1 mg is dissolved in 20 μl in DMSO. The stock solution is diluted with PBS 1:105.

U0126 (M=380.49 g/mol): 1 mg of inhibitor is dissolved in 100 μl of DMSO. The stock solution is diluted with PBS 1:20.

The wells of the 48-well plate are supplied with the following amounts of inhibitor:

| | U0126 | | | | SB203580 | | | |
|---|---|---|---|---|---|---|---|---|
| | F | | E | | D | | C | |
| 1 | 0 μM | (0 μl) | 0 μM | (0 μl) | 0 μM | (0 μl) | 0 μM | (0 μl) |
| 2 | 5 μM | (1 μl) | 5 μM | (1 μl) | 5 μM | (1 μl) | 5 μM | (1 μl) |
| 3 | 10 μM | (2 μl) | 10 μM | (2 μl) | 10 μM | (2 μl) | 10 μM | (2 μl) |
| 4 | 25 μM | (5 μl) | 25 μM | (5 μl) | 20 μM | (4 μl) | 20 μM | (4 μl) |
| 5 | 50 μM | (10 μl) | 50 μM | (10 μl) | 30 μM | (6 μl) | 30 μM | (6 μl) |
| 6 | 75 μM | (15 μl) | 75 μM | (15 μl) | 40 μM | (8 μl) | 40 μM | (8 μl) |
| 7 | 0 μM | (0 μl) | 0 μM | (0 μl) | 0 μM | (0 μl) | 0 μM | (0 μl) |
| 8 | 0 μM | (0 μl) | 0 μM | (0 μl) | 0 μM | (0 μl) | 0 μM | (0 μl) |

Incubation is then carried out in an incubator for 2 hours. The lipoplexes are then prepared. For that purpose, 1 μl of anti-luciferase-siRNA stock solution is pipetted into 300 μl of PBS and mixed by gentle pipetting up and down. Of that solution, only 240 μl are used further. 1 μl of non-specific-control-siRNA is pipetted into 300 μl of PBS and mixed by gently pipetting up and down. Of that solution, only 40 μl are used further.

3 μl of Metafectene Pro are pipetted into 225 μl of PBS and likewise mixed by gently pipetting up and down. Then 190 μl are pipetted into the anti-luciferase-siRNA working solution and 35 μl into the non-specific-control-siRNA working solution. The solutions are mixed and incubated for 15 min. Lipoplexes having an siRNA reagent ratio of 1:5 μg/μl are obtained.

Finally, 15 μl portions of the anti-luciferase-siRNA-lipoplex solution are pipetted into the first 6 rows of the plate. 15 μl of the non-specific-control-siRNA-lipoplex solution are introduced into the seventh row of the plate. An siRNA amount of 1 pMol/well is obtained. Row 8 remains untransfected. Incubation is then carried out in a CO₂ incubator (10%) for 24 hours.

3rd Day:

On the third day the medium of all wells is renewed and the amounts of inhibitor indicated in the table replaced. The steps of lipoplex preparation and transfection are repeated for columns E and C after the addition of inhibitor. Incubation is then carried out in a CO₂ incubator (10%) for a further 24 hours.

4th Day:

Luciferase Assay and BCA Protein Assay:

Both assays are carried out in accordance with the manufacturer's instructions.

Evaluation:

The medium is removed from the 48-well plate and the cells are washed with 100 μl of PBS. 120 μl of luciferase-lysis buffer are then added. The plate is incubated on ice for about 30 minutes and mixed for 20 to 30 seconds by careful agitation (tapping). 25 μl are then removed from each well of that 48-well plate and introduced, with an analogous set-up, into a second 48-well plate. The luciferase assay is carried out with the first plate and the BCA test is carried out with the second plate.

Luciferase Assay:

Parameter Settings of the Luminometer:
measuring time: 15 sec
time interval between solution injections: 0.5 sec
interval between solution injection and measurement: 0.1 sec

BCA Protein Assay:
evaluation at wavelength 562 nm

All measurements were carried out three times. In the case of the luciferase assay three times with three new cell extracts. The values obtained from the luciferase assay (RLU) were standardised to 1 μl of cell extract and 1 sec measuring time. The values obtained from the BCA protein assay were likewise standardised to 1 μl of cell extract.

Luciferase Assay:

RLU/μl ell extract/sec

Average Value of 3×3 Measurements:

| | F | E | D | C |
|---|---|---|---|---|
| 1 | 1157 | 1001 | 1027 | 976 |
| 2 | 375 | 641 | 693 | 696 |
| 3 | 567 | 162 | 747 | 656 |
| 4 | 268 | 179 | 630 | 617 |
| 5 | 193 | 171 | 671 | 587 |
| 6 | 260 | 103 | 669 | 610 |
| 7 | 1829 | 2290 | 2029 | 2135 |
| 8 | 1674 | 2249 | 2504 | 2531 |

BCA Assay:

ABS/μl cell extract

Average Value of 3 Measurements:

| | F | E | D | C |
|---|---|---|---|---|
| 1 | 0.0196 | 0.0196 | 0.0192 | 0.0192 |
| 2 | 0.0192 | 0.0204 | 0.0193 | 0.0189 |
| 3 | 0.0189 | 0.0196 | 0.0193 | 0.0187 |
| 4 | 0.0183 | 0.0188 | 0.0186 | 0.0186 |
| 5 | 0.0182 | 0.0191 | 0.0183 | 0.0186 |
| 6 | 0.0182 | 0.0190 | 0.0184 | 0.0192 |
| 7 | 0.0184 | 0.0186 | 0.0183 | 0.0184 |
| 8 | 0.0184 | 0.0178 | 0.0181 | 0.0187 |

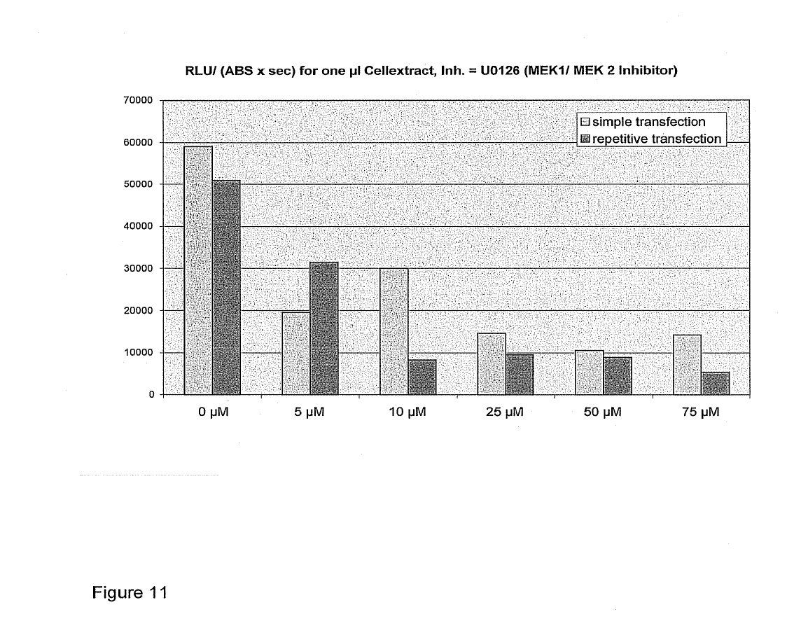

See FIG. 11

### Example 9

siRNA transfection in the presence of TLR7/8 agonists:
imiquimod, InvivoGen, Cat. No.: tirl-imq
ssRNA40/LyoVec, InvivoGen, Cat. No.: tirl-lrna-40

Detailed Description of Experiment:

1st Day:

Preparation of the Cells:

The cells are to be plated out at a cell count of 2×10⁴ cells/cm² per well of 250 μl of complete medium.

Preparation of the Lipoplexes:

First of all, the first 5 wells of the first three rows of a 48-well plate are each filled with 30 μl of PBS.

The stock solution of anti-luciferase-siRNA and the non-specific-control-siRNA is adjusted to a concentration of 1 pmol/10 μl with PBS. Then 10 μl of the working solution of the anti-luciferase-siRNA are pipetted into each of the first three wells of the first three rows of the 48-well plate. 10 μl of the working solution of the non-specific-control-siRNA are pipetted into each fourth well.

The Metafectene Pro reagent is diluted 1:300 with PBS. 20 μl of that dilution are introduced into each well containing siRNA.

The lipoplexes are then incubated for 20 minutes at room temperature. Then 250 μl of cell suspension are added to each well.

The second plate is charged analogously.

Addition of the Agonists:

Two different agonists are added to the first 2 columns of the two 48-well plates, with three different working concentrations of the agonists being used. The agonists are in lyophilised form. They are dissolved with endotoxin-free water to form stock solutions having the concentration 100 μg/ml. The stock solutions are each diluted 1:10 with water to give working solutions.

Of the imiquimod working solution, 0.78 μl (=0.25 μg/ml final concentration) are pipetted into A1, 15.5 μl (=5 μg/ml) into A2 and 31 μl (=10 μg/ml) into A3.

Of the ssRNA40/LyoVec working solution, 0.78 μl (=0.5 μg/ml final concentration ssRNA40) are pipetted into B1, 15.5 μl (=5 μg/ml) into B2 and 31 μl (=10 μg/ml) into B3. After the addition of the agonists, the two 48-well plates are incubated in a CO₂ incubator for 48 hours.

Day 3:

Vitality Determination:

After an incubation period of 48 hours, the cells of a plate are trypsinised and, using a Neubauer counting chamber, vitality measurement is carried out for each well with trypan blue.

Luciferase Assay and BCA Protein Assay: Analogous to Example 13

Results

Trypan blue staining

Between 40 and 70 cells were evaluated.

| | Vitality [%] |
|---|---|
| Imiquimod 0.5 μg/ml + | 98.6 |
| anti-luciferase-siRNA/Metafectene Pro | |
| Imiquimod 5 μg/ml + | 88.7 |
| anti-luciferase-siRNA/Metafectene Pro | |
| Imiquimod 10 μg/ml + | 88.6 |
| anti-luciferase-siRNA/Metafectene Pro | |
| ssRNA40/LyoVec 0.5 μg/ml + | 97.8 |
| anti-luciferase-siRNA/Metafectene Pro | |
| ssRNA40/LyoVec 5 μg/ml + | 81.5 |
| anti-luciferase-siRNA/Metafectene Pro | |
| ssRNA40/LyoVec 10 μg/ml + | 83.0 |
| anti-luciferase-siRNA/Metafectene Pro | |
| Anti-luciferase-siRNA/Metafectene Pro | 87.7 |
| Non-specific-control-siRNA/Metafectene Pro | 92.6 |
| Blind sample | 95.9 |

Luciferase Assay:

RLU/μl cell extract/sec

Average Value of 3×3 Measurements:

| | 1 | 2 | 3 | 4 | 5 |
|---|---|---|---|---|---|
| A | 676 | 1023 | 1604 | 3016 | 2936 |
| B | 349 | 223 | 1302 | 2753 | 2673 |
| C | 116 | 98 | 1481 | 2822 | 2476 |

BCA Assay ABS/μl Cell Extract

Average Value of 3×3 Measurements:

| | 1 | 2 | 3 | 4 | 5 |
|---|---|---|---|---|---|
| A | 0.0086 | 0.0080 | 0.0080 | 0.0087 | 0.0093 |
| B | 0.0069 | 0.0068 | 0.0078 | 0.0080 | 0.0092 |
| C | 0.0073 | 0.0069 | 0.0078 | 0.0080 | 0.0088 |

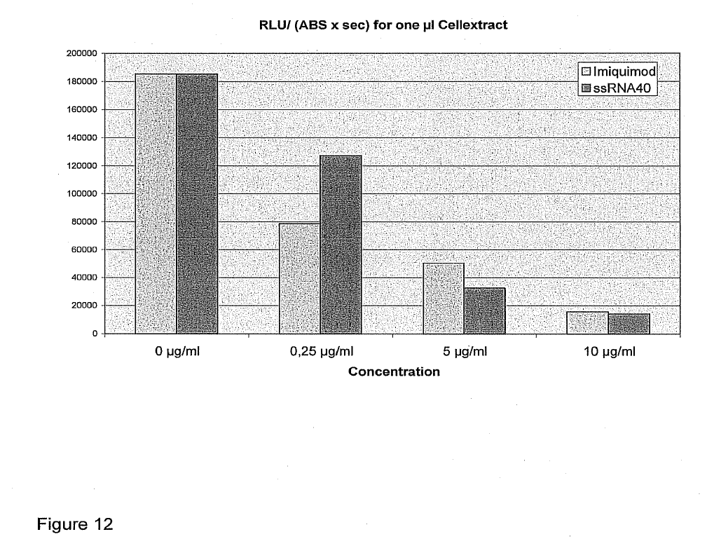

See FIG. 12

The results of the Examples according to the invention also allow the conclusion to be drawn that not only the genetic material but also the chemical agents, such as, for example, cationic lipids and cationic polymers, are detected by the TLRs. In accordance with the present results, it must be assumed that in the case of Hela cells, Metafectene Pro as transfection reagent and high-quality DNA (plasmid), the following TLRs are involved. TLR 4 detects traces of LPS in the plasmid solution. TLR9 and TLR3 detect genetic material, that is to say in this case plasmids or corresponding impurities. TLR1 and TLR2 detect the transfection reagent.

A possible but non-committal explanation may therefore also be that different cells exhibit optimum results with different ratios of genetic material and transfection reagent. Cells which, for example, express very large numbers of TLR9 and few TLRs for the reagents should accordingly exhibit optimum results at a ratio in which a small amount of DNA encounters a large amount of reagent.

It is evident that the activation of the TLRs before and during transfection depends upon a large number of factors. Depending upon the expression profile of the target cell, for example, the cells may be particularly sensitive to certain PAMPs. Furthermore, different reagents and different genetic material can activate different TLRs. Even different DNA sequences can have an effect on the transfection results, depending upon the CpG content. Last but not least, impurities also play a role that is not to be underestimated. For example, DNA of bacterial origin can be contaminated with, for example, LPS or flagellin or RNA. ssRNA can be contaminated with dsRNA and thus, in addition to TLR7/8, TLR3 also respond and vice versa. Different treatment of the cells can lead to different results as a result of stress signal transduction cascades. In summary, it can be said that for different transfection experiments different blocking strategies are required.

The different transfection results for blocking of the innate immune system in respect of single and repetitive transfection can likewise be interpreted. It is known that endocytosis, that is to say the external membrane transport, is regulated to a great extent by kinases. By means of the fast route of phosphorylation, on detection of pathogenically associated patterns the cell can down-regulate endocytosis and thus close a gateway for pathogens. Because the signal transduction cascades also transmit the signal via kinases, there can be assumed to be a connection here. Significant increases in transfection efficiency as early as on first transfection indicate that rapid defence processes have been suppressed, that is to say presumably also membrane transport. Defence processes that proceed via the expression of special proteins or cytokines take considerably longer and are effective within a time frame only applicable to double transfection. An increase in transfection efficiency after one day as a result of repetitive transfection points to reduced expression of one or more proteins that are important for the defence process.

Only in the case of transfection of siRNA is it possible to achieve an improvement in the transfection result with an activation of TLRs, because in this case the necessary RNAi machinery is a component of the innate immune system. In this case, however, blocking of various signal transduction pathways (Example U0126) can also be advantageous.

As already mentioned, the invention can be used for treatment purposes. In particular, the invention can be used for the gene therapy of, for example, cystic fibrosis, muscular dystrophy, phenylketonuria, maple syrup disease, propionazidaemia, methylmalon-azidaemia, adenosine deaminase deficiency, hypercholesterolaemia, haemophilia, β-thalassaemia and cancer. Gene-therapeutic treatment methods are also of interest when hormones, growth factors, cytotoxins, or proteins having an immunomodulating action are to be synthesised in the organism. For the above-mentioned purposes, by means of the invention DNA fragments can be introduced effectively into cells in which such DNA is able to display the desired action without resulting in undesirable side-effects. The desired action can be the replacement of missing or defective DNA regions or the inhibition of DNA regions (for example by antisense-DNA/RNA or siRNA) that trigger the disease, in the diseased cell type. In that way, it is possible, for example, for tumour-suppressing genes to be used in cancer therapy or a contribution to be made towards preventing cardiac and vascular diseases by the introduction of genes that regulate cholesterol. Furthermore, DNA that encodes ribozymes, siRNA or shRNA or the ribozymes or siRNA themselves can be inserted into diseased cells. The translation of DNA produces active ribozymes or siRNA which cleave m-RNA catalytically at specific sites and in that way prevent transcription. In that way, for example, viral m-RNA can be cleaved without affecting other cellular m-RNA. The replication cycle of viruses (HIV, herpes, hepatitis B and C, respiratory syncytial virus) can be interrupted in that way. Other diseases that are said to be cured specifically by means of treatment with siRNA are age-related macular degeneration (eye disease), cancer of the liver, solid tumours, amyotrophic lateral sclerosis and inflammatory diseases. Transfection is playing an ever increasing role also in cancer treatment, for example for the preparation of cancer vaccines, and so that too is a possible field of application for the invention.

The invention can also be used, for example, in vaccination methods which function on the basis of the expression of DNA that encodes immunogenic peptides in the human or animal body. For that purpose, for example, lipid/DNA complexes are used as vaccines. The insertion of the DNA into the cells of the body results in expression of the immunogenic peptide and thus triggers the adaptive immune response.

The following definitions are given according to the invention by way of example but are on no account limiting:

Transfection:

Insertion of genetic material into a eukaryotic cell.

Transfection Result/Transfection Efficiency

The amount of a protein expression of a cell population as a result of transfection processes with genetic material which inter alia encodes that expressed protein, or the extent of a knock-down of a protein expression of a cell population as a result of transfection processes with genetic material that is able to trigger such a knock-down, especially siRNA or ribozymes or DNA that codes for shRNA or ribozymes, or the proportion of the cells of a total population of cells that exhibits the biological activity of the inserted genetic material as a result of transfection processes. At the same time, the physiological state of the cell population should be affected as little as possible, that is to say the protein expression profile of the cell population should ideally be changed only in respect of the proteins the genes of which have been inserted into the cell or the expression of which is to be reduced or prevented by the inserted genetic material.

Non-Viral Gene Delivery System:

Non-viral gene delivery systems are not generated by recombination of genetic material of naturally occurring viruses. They are capable of inserting genetic material into eukaryotic cells. Non-viral gene delivery systems are especially physical methods and chemical methods. Physical methods localise at least the genetic material in the vicinity of the cell; in particular physical methods, however, utilise energy supply especially in the form of thermal, kinetic, electrical or other energy to mediate transport of the genetic material through the cell membrane. Chemical methods are based either on a chemical modification or derivatisation of the nucleic acids, which especially render them cell-permeable, or consist especially of substances that bind DNA and are able to mediate transport through the cell membrane. In particular, they use electrostatic forces or hydrogen bridge bonds for binding the nucleic acids. In turn, the transport of the DNA through the cell membrane takes place especially as a result of an active transport mechanism of the cell, i.e. endocytosis. Substances having those properties contain especially cationic lipids, cationic polymers, cationic peptides or molecules having a domain that is able to bind DNA or RNA and at the same time have a second domain containing a ligand which is recognised by a receptor also of the cell surface and triggers endocytosis as a result of that recognition process. A second possibility is that the ligand is capable of triggering membrane transfer, that is to say of mediating transport to the other side of the membrane. The substances can also be specially formulated, especially in the form of micelles or liposomes, and may also consist of a plurality of components, especially having different functions.

Gene Therapy

Therapy for curing or alleviating diseases in which modified or unmodified nucleic acids are used as active substance.

Innate Immunity

Innate immunity is distinguished from acquired or adaptive immunity in that it protects against a causative organism without needing ever to have previously come into contact with the causative organism in order to educate the immune system. Innate immunity is an inherent property of most cell types and consists of two parts, the intracellular component and the intercellular component. The intracellular component utilises receptors' recognition of molecular structures that are attributable to pathogens. Such receptors stimulate especially signal transduction cascades which, especially by expression of a large number of cell-endogenous genes and phosphorylation of important proteins, result in a change in the physiological state (for example “antiviral state”) of the cells directly involved. In addition, cytokines are secreted.

By means of the intercellular component of the innate immune system, affected cells inform unaffected cells by way of those messenger substances (cytokines) and also trigger therein a modified physiological state (for example “antiviral state”), the cytokines docking onto cytokine receptors of the other cells and in turn triggering a signal transduction cascade. The intercellular component is therefore distinguished from the intracellular component by the different receptors (cytokine receptors instead of PRRs (pattern recognition receptors) and agonists (cytokines instead of pathogenic patterns)).

Antiviral State

State of cells which is distinguished by the fact the cell attempts to prevent the possible biological activity of foreign genetic material by counter-measures.

Transfection In Vivo

The insertion of genetic material into eukaryotic cells takes place in a living organism.

Transfection In Vitro

The insertion of genetic material into eukaryotic cells takes place outside a living organism, especially in vessels which are suitable for culturing eukaryotic cells.

Genetic Material

Nucleic acids, especially ribonucleic acids or deoxyribonucleic acids, which consist especially of two at least partially complementary strands (double-stranded=ds), for example dsDNA and dsRNA, or which consist especially of one strand (single-stranded=ss), for example ssDNA and ssRNA, which may have partially complementary regions which can be joined to one another by means of hydrogen bridge bonding. In the case of DNA, the genetic material serves for the production of RNA and/or proteins. In the case of ssRNA, it serves for the production of proteins. In the case of dsRNA, the genetic material serves to achieve a knock-down of a gene by RNA-interference.

Modified Genetic Material

Natural nucleic acids the properties of which have been changed by modification. Those modifications can be, especially, chemical changes which relate especially to the phosphate structure, the sugars or bases, which is intended to increase especially the stability of the nucleic acids towards nucleases and ribonucleases. Furthermore, molecules (labels) can be covalently or non-covalently attached to the nucleic acids, resulting in new properties of the nucleic acids, especially in the ability to be monitored optically by means of fluorescent labels or labels which direct the nucleic acids to a specific site in the cell (localisation elements) or labels which mediate the passage of nucleic acids through membranes and so render nucleic acids, for example, cell-permeable.

An example of a modification is the exchange of oxygen for sulphur in the phosphate structure, the methylation of 2′-OH groups of the ribose in the case of RNA or the complete substitution of 2′-OH groups by fluorine in order to increase stability towards nucleases. A further example is the attachment of FITC (fluorescein isothiocyanate) as a fluorescent label in order to be able to monitor the path of the genetic material in the cell microscopically or the attachment of so-called NLS (nuclear localisation signals, for example PKKKRKVG) in order to effect transport into the cell nucleus.

siRNA (Short Interfering RNA):

Short dsRNA (up to 28 bp) which is able to bring about the knock-down of a protein by RNA-interference.

shRNA (Short Hairpin RNA):

Short ssRNA which has complementary regions at the 3′-end and at the 5′-end and as a result is able to hybridise by means of hydrogen bridge bonding and form a hairpin structure. shRNA is able to bring about the knock-down of a protein by RNA-interference.

Receptor:

Molecule that is capable of detecting a substance (agonist) and thus triggers a biological reaction; receptors are especially proteins.

Blocking:

Prevention of the biological function, especially of proteins.

Suppression:

Interruption of the communication network of the immunity, that is to say of one or more signal transduction cascade(s) triggering an immune response, especially the innate intracellular and/or intercellular immunity. As a result of that interruption, the immunity is weakened, with the result that the immune response is reduced.

DNA/RNA-Binding Domain:

Region in a molecule which carries DNA bound covalently or by means of non-covalent interactions; in particular, the non-covalent interactions are electrostatic forces and hydrogen bridge bonds.

Signal Transduction Cascade:

Starting from a receptor which detects the presence of a substance by attachment of that substance to the receptor, the information relating to the presence of that substance is converted into a signal and transmitted by way of a chain of molecules by signal transduction. At the end there is a biological reaction. In particular, the signal produced by the receptor is taken up by adapter molecules and transmitted, especially by way of kinases, especially to transcription factors. The transcription factors stimulate the expression of genes that mediate the biological reaction.

Knock-Down:

Weakening or switching-off of the translation of an mRNA to a protein during protein biosynthesis.

Antibodies:

Molecules, especially proteins, that are capable of recognising molecular structures, especially other proteins, and by binding affect the biological action thereof. In the context of the invention they are blocking antibodies, that is to say in the case of the receptors of the innate immune system the signal transmission is reduced or prevented. The antibodies can be polyclonal or monoclonal. The antibodies can have been humanised. As a rule, they have to be directed against the receptor of a species the cells of which are to be transfected. As a result of the similarity of the receptors, however, it can also happen that an antibody is cross-reactive. Furthermore, the antibodies can have been modified, that is to say, for example, parts of molecules can have been split off (for example Fc fragments, so that only Fab fragments remain), provided that the biological action is not appreciably reduced thereby.

Furthermore, it is additionally possible to attach to the antibody parts of molecules that impart additional properties to the antibody, provided that the biological action is not appreciably reduced thereby.

An example is the attachment of fluorescent labels or the attachment of hydrophobic radicals (for example phospholipids, A. Schnyder et al.; J. Am. Soc. for Exp. Neuro-Therapeutics; 2005; 2; 99-107) in order to facilitate anchoring in membranes and/or liposomes.

Intrabodies:

Intracellularly expressed antibodies.

Aptamers:

RNA molecules which, by formation of a tertiary structure, are able to recognise molecular structures, especially proteins, similarly to an antibody and by binding affect the biological action thereof. Aptamers can consist of modified or unmodified RNA or DNA.

Antagonist:

Substance that inhibits an agonist by blocking a corresponding receptor in its action, without itself triggering an effect.

Agonist:

An agonist is able to achieve a biological action by binding to a receptor.

Antiviral Cytokines:

Cytokines which are expressed in the event of infection of a eukaryotic cell with viruses. Examples are interferon alpha, interferon beta, interferon gamma, TNF alpha, TFN beta, interleukin 6, 8, 15, 28 and 29.

Inhibitors:

Molecules that are able to inhibit the biological action of another molecule, especially of proteins. In particular, the inhibitors are themselves proteins, modified or unmodified nucleic acids or small organic molecules.

TLRs, RIG-I-Helicase, RIG-I-Like Helicase:

Receptors which are combined across species into groups according to their function.

Adapter Molecules:

Molecules which receive a signal from receptors and which are combined across species into groups according to their function.

### ABBREVIATIONS

AP-1=Activated Protein-1

ERK=Extracellular-signal Regulated Kinase

IkB=Inhibitory-binding protein kB

IKK=IkappaBKinase=inhibitory-binding protein KB kinase

IKKa=Ikkalpha=I Kappa Kalpha=IKK1

IKKb=IKKbeta=I Kappa Kbeta=IKK2

IKKd=IKKdelta=1 Kappa Kdelta

IKKe=IKKepsilon=I Kappa Kepsilon

IKKg=IKK gamma=I Kappa Kgamma=IKK3

IKKi=IKK epsilon

IRAK1=Interleukin 1 Receptor-Associated Kinase 1

IRAK4=interleukin-1 receptor-associated kinase 4

IRF=Interferon regulating Factor

JNK=c-Jun N-terminal Kinase

JAK=Janus activated Kinase

Mal=TIRAP=MyD88-adapter-like

MAPK=Mitogen activated Protein Kinase

MEK=MAPK/ERK kinase

MKK=MAP Kinase Kinase

MKK=Mitogen-activated protein kinase kinase

MSK=Mitogen and stress activated kinase

MyD88=Myeloid differentiation factor 88

NAP1=Nck-associated protein 1

NEMO=IKK gamma

NF-kB=Nuclear Factor kappaB

P1 3K=Phosphoinositol-3-Kinase

PKR=Protein Kinase R=Protein Kinase RNA-activated

PKB=Protein Kinase B

PDK1=Phosphoinositide-dependent Protein Kinase 1

PDK2=Phosphoinositide-dependent Protein Kinase 1

Rac1=Ras-related C3 botulinum toxin substrate 1

RIP1=Receptor-interacting protein 1

STAT=Signal Transducers and Activators of Transcription

TAK1=Transforming growth factor-β-activated kinase

TBK1=IKKd=TANK-binding Kinase

TIRAP=Mal=Toll-interleukin 1 receptor (TIR) domain-containing adapter protein

TRAF3=TNF receptor-associated factor 3

TRAF6=TNF receptor-associated factor 6

TRAM=TRIF-related adaptor molecule

TRIF=Toll/IL-1 receptor domain-containing adaptor inducing interferon-b adaptor protein

1-33. (canceled)

34. A composition for a transfection, comprising:
a) a non-viral gene delivery system, the non-viral gene delivery system comprising
(i) a cationic lipid, a cationic polymer or a cationic protein; and/or
(ii) a compound which has a DNA- and/or RNA-binding domain and is able to trigger receptor-mediated endocytosis or a membrane transfer; and/or
(iii) a compound which is covalently bound to DNA and/or RNA and is able to trigger receptor-mediated endocytosis or a membrane transfer; and
b) a composition for at least partially suppressing and/or activating the innate intracellular and/or intercellular immunity, selected from:
(i) an antibody to TLR 1, TLR 2, TLR 3, TLR 4, TLR 5, TLR 6, TLR 7, TLR 8, TLR 9, TLR 10, TLR 11, TLR 12 or TLR 13;
(ii) an antibody to a cytokine receptor or a cytokine receptor antagonist;
(iii) an inhibitor of kinase MEK1 and/or MEK2;
(iv) an agonist for TLR7 and/or TLR8, selected from the group comprising bropirimine (2-amino-5-bromo-6-phenyl-4-pyrimidinone), imidazoquinolines, thiazoloquinolines and guanosine analogues; and
(v) a combination thereof.

35. A kit for transfection, comprising:
a) a non-viral gene delivery system, the non-viral gene delivery system comprising
(i) a cationic lipid, a cationic polymer or a cationic protein; and/or
(ii) a compound which has a DNA- and/or RNA-binding domain and is able to trigger receptor-mediated endocytosis or a membrane transfer; and/or
(iii) a compound which is covalently bound to DNA and/or RNA and is able to trigger receptor-mediated endocytosis or a membrane transfer; and
b) a composition for at least partially suppressing and/or activating the innate intracellular and/or intercellular immunity, selected from:
(i) an antibody to TLR 1, TLR 2, TLR 3, TLR 4, TLR 5, TLR 6, TLR 7, TLR 8, TLR 9, TLR 10, TLR 11, TLR 12 or TLR 13;
(ii) an antibody to a cytokine, receptor or a cytokine receptor antagonist;
(iii) an inhibitor of kinase MEK1 and/or MEK2;
(iv) an agonist for TLR7 and/or TLR8, selected from the group comprising bropirimine (2-amino-5-bromo-6-phenyl-4-pyrimidinone), imidazoquinolines, thiazoloquinolines and guanosine analogues; and
(v) a combination thereof.

36. A composition or kit according to claim 34 or claim 35, wherein the non-viral gene delivery system comprises a cationic lipid.

37. A composition or kit according to claim 34 or 35 wherein the non-viral gene delivery system defined in a) comprises a cationic lipid having the following formula: 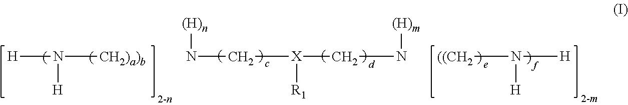
wherein
R₁ is 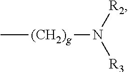
wherein
R₂′ and R₃ are each independently of the other dodecyl, dodecenyl, tetradecyl, tetradecenyl, hexadecyl, hexadecenyl, octadecyl, octadecenyl or other alkyl radicals which, in all possible combinations, are saturated, unsaturated, branched, unbranched, fluorinated or non-fluorinated and are composed of from 5 to 30 carbon atoms;
X is 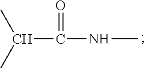
and wherein
m=0 and n=0; or
m=0 and n=1; or m=0 and
D=2; or
m=1 and n=1; or m=1
and n=2; or
m=2 and n=2; and
g is 1, 2, 3, 4, 5, 6, 7 or 8;
a is 0, 1, 2.3. 4′ or 6;
b is 0, 1, 2, 3, 4, 5 or 6;
c is 0, 1, 2, 3, 4, 5 or 6;
d is 0, 1, 2, 3, 4, 5 or 6;
e is 0, 1, 2, 3, 4, 5 or 6, and
f is 0, 1,2, 3, 4, 5 or 6.

38. A composition or kit according to claim 37 wherein R₂ and R₃ are each independently of the other dodecyl, dodecenyl, tetradecyl, tetradecenyl, hexadecyl, hexadecenyl, octadecyl, octadecenyl;
m and n are 1;
g is 1, 2, 3, 4, 5, 6, 7 or 8;
a is 0, 1, 2, 3, 4, 5 or 6;
b is 0, 1, 2, 3, 4, 5 or 6;
c is 0, 1, 2, 3, 4, 5 or 6;
d is 0, 1, 2, 3, 4, 5 or 6;
e is 0, 1, 2, 3, 4, 5 or 6, and
f is 0, 1,2, 3, 4, 5 or 6.

39. A composition or kit according to claim 34 or 35 wherein the composition or kit of parts comprises modified or unmodified genetic material, especially modified or unmodified ssDNA, modified or unmodified dsDNA, modified or unmodified ssRNA, modified or unmodified dsRNA and/or modified or unmodified siRNA.

40. A composition or kit according to claim 34 or 35 wherein the composition for at least partially suppressing and/or activating the innate intracellular and/or intercellular immunity is 1,4-diamino-2,3-dicyano-1,4-bis(o-aminophenylmercapto)butadiene (U0126); imiquimod (R837, 1-(2-methylpropyl)-1H-imidazo[4,5-c]quinoline-4-amine); resiquimod (R848, 4-amino-2-(ethoxymethyl)-a,a-dimethyl-1H-imidazo[4,5-c]quinoline-1-ethanol); gardiquimod (1-(4-amino-2-ethylaminomethylimidazo[4,5-c]quinolin-1-yl)-2-methylpropan-2-ol); CL075; CL097; loxoribine (7-allyl-7,8-dihydro-8-oxo-guanosine); isatoribine (7-thia-8-oxoguanosine); bropirimine (2-amino-5-bromo-6-phenyl-4-pyrimidinone); or any combination thereof.

41. A composition or kit according to claim 34 or 35 wherein the composition for at least partially suppressing and/or activating the innate intracellular and/or intercellular immunity is an antibody to TLR 3, TLR 7, TLR 8 or TLR 9.

42. A composition or kit according to claim 34 or 35 wherein the composition for at least partially suppressing and/or activating the innate intracellular and/or intercellular immunity is an antibody or antagonist against interleukin-1-receptors, especially IL-ra; interferon-type-I-receptors; interferon-gamma-receptors;
or tumour necrosis factor receptors.

43. A kit according to claim 35 wherein
(i) all components are present entirely separately from one another;
(ii) components a) and b) are present separately from one another; or
(iii) components a) and b) are present together.

44. A pharmaceutical composition comprising a composition according to claim 34.

45. A pharmaceutical kit comprising a kit according to claim 35.

46. A method for improving the transfection result of non-viral gene delivery systems, comprising:
a) the cells are treated before and/or during transfection with at least one means for at least partially suppressing the innate intracellular and/or intercellular immunity and, during transfection, genetic material, especially modified and/or unmodified ssDNA, modified and/or unmodified dsDNA, modified and/or unmodified ssRNA, modified and/or unmodified dsRNA and/or modified and/or unmodified siRNA, is introduced into the cells; or
b) the cells are treated before and/or during and/or after transfection with at least one means for at least partially activating the innate intracellular and/or intercellular immunity and, during transfection, modified and/or unmodified siRNA is introduced into the cells.

47. A method according to claim 46 wherein the non-viral gene delivery system
(i) comprises a cationic lipid, a cationic polymer or a cationic protein; and/or
(ii) comprises a compound which has a DNA- and/or RNA-binding domain and is able to trigger receptor-mediated endocytosis or a membrane transfer; and/or
(iii) comprises a compound which is covalently bound to DNA and/or RNA and is able to trigger receptor-mediated endocytosis or a membrane transfer; and/or
(iv) is based on a physical method such as electroporation, microinjection, magnetofection, ultrasound or a ballistic or hydrodynamic method.

48. A method according to claim 46 wherein the cells are treated up to 4 days before transfection with the at least one means for at least partially suppressing or activating the innate intracellular and/or intercellular immunity.

49. A method according to claim 46 wherein the cells are simultaneously treated with the at least one means for at least partially suppressing or activating the innate intracellular and/or intercellular immunity and brought into contact with the non-viral gene delivery system.

50. A method according to claim 46 wherein the at least one means for at least partially suppressing the innate intracellular and/or intercellular immunity comprises an antibody, intrabody, aptamer, antagonist, inhibitor and/or an siRNA.

51. A method according to claim 46 wherein by knock-down with siRNA, at least one gene that codes for a protein necessary for signal transduction via TLR is switched off.

52. A method according to claim 46 wherein the innate intracellular and/or intercellular immunity is at least partially suppressed by blocking of at least one of the group TLR 1, TLR 2, TLR 3, TLR 4, TLR 5, TLR 6, TLR 7, TLR 8, TLR 9, TLR 10, TLR 11, TLR 12, TLR 13, CD14, CD38, RIG-I helicase and RIG-I-like helicase, especially by blocking of at least one of the group TLR 1, TLR 2, TLR 4 and TLR 9.

53. A method according to claim 46 wherein the innate intracellular and/or intercellular immunity is at least partially suppressed by blocking of at least one kinase from the group MEK1 and MEK2.

54. A method according to claim 53 wherein the kinase(s) MEK1 and/or MEK2 is/are blocked by 1,4-diamino-2,3-dicyano-1,4-bis(o-aminophenylmercapto)-butadiene (U0126).

55. A method according to claim 46 wherein the innate intercellular immunity is at least partially suppressed by blocking of at least a cytokine, a tumour necrosis factor, an interleukin or an interferon.

56. A method according claim 55 wherein an interferon of type I, especially an interferon from the group interferon-alpha, interferon-beta, interferon-gamma and interferon-omega, is blocked.

57. A method d according to claim 46 wherein the innate intercellular immunity is at least partially suppressed by blocking of at least one receptor from the group of the cytokine receptors, interferon receptors, especially receptors for interferons of type I, interleukin receptors and tumour necrosis factor receptors.

58. A method according to claim 46 wherein the means for activating the innate immunity is an agonist.

59. A method according to claim 46 wherein the innate intracellular and/or intercellular immunity is at least partially activated by at least one agonist for a TL receptor, especially by at least one agonist for TLR7 and/or TLR8.

60. A method according to claim 59 wherein the at least one agonist for TLR7 and/or TLR8 is selected from the group comprising bropirimine (2-amino-5-bromo-6-phenyl-4-pyrimidinone), imidazoquinolines, thiazoloquinolines, guanosine analogues and ssRNA.

61. A method according to claim 60 wherein the at least one agonist is
(i) imiquimod (R837, 1-(2-methylpropyl)-1H-imidazo[4,5-c]quinoline-4-amine), resiquimod (R848, 4-amino-2-(ethoxymethyl)-a,a-dimethyl-1H-imidazo[4,5-c]quinoline-1-ethanol) or gardiquimod (1-(4-amino-2-ethylaminomethylimidazo[4, 5-c]quinolin-1-yl)-2-methylpropan-2-ol); or
(ii) CL075 or CL097; or
(ii) loxoribine (7-allyl-7,8-dihydro-8-oxo-guanosine) or isatoribine (7-thia-8-oxo-guanosine); or
(iv) ssRNA having U-rich and/or GU-rich sequences, especially ssRNA having the sequence motifs UGUGU and/or GUCCUUCAA.

62. A method according to claim 46 wherein the innate intercellular immunity is at least partially activated by at least one agonist for receptors of antiviral cytokines, especially by interferon-beta or interferon-gamma.

63. A method according to claim 46 wherein the cells are treated up to 2 days after transfection with the composition for at least partially activating the innate intracellular and/or intercellular immunity.

64. A method according to claim 46 wherein the non-viral gene delivery system comprises a cationic lipid according to claim 37.

65. A method for treating a subject suffering from or susceptible to a disease, comprising:
administering a composition of claim 34 to the subject to thereby treat the disease.

66. A method of claim 65 wherein the subject is treated by gene therapy.

67. The method of claim 65 wherein the subject is suffering from or susceptible to a disease of cystic fibrosis, muscular dystrophy, phenylketonia, maple syrup disease, propionazidaemia, methylmalonazidaemia, adenosine deaminase deficiency, hypercholesterolaemia, haemophilia, β-thalassamia, cancer, a viral disease, macular degeneration, amyotrophic lateral sclerosis and/or an inflammatory disease.

68. The method of claim 65 wherein the subject is suffering from a disease of cystic fibrosis, muscular dystrophy, phenylketonia, maple syrup disease, propionazidaemia, methylmalonazidaemia, adenosine deaminase deficiency, hypercholesterolaemia, haemophilia, β-thalassamia, cancer, a viral disease, macular degeneration, amyotrophic lateral sclerosis and/or an inflammatory disease.


SEQUENCE LISTING (5 sequences)

SEQ ID NO: 1 - RNA, 9 nt, Homo sapiens
guccuucaa

SEQ ID NO: 2 - DNA, 21 bp, Photinus pyralis
cuuacgcugaguacuucgatt

SEQ ID NO: 3 - DNA, 21 bp, Photinus pyralis
ucgaaguacucagcguaagtt

SEQ ID NO: 4 - DNA, 21 bp, Artificial
  siRNA; non-specific control sequence; sense
agguaguguaaucgccuugtt

SEQ ID NO: 5 - DNA, 21 bp, Artificial
  siRNA; non-specific control sequence; antisense
caaggcgauuacacuaccutt